(19) United States
(12) Patent Application Publication
(10) Pub. No.: US 2026/0146238 A1
(43) Pub. Date: May 28, 2026

(54) UREA-BASED ICE RECRYSTALLIZATION INHIBITOR COMPOUNDS AND COMPOSITIONS, AND USES THEREOF FOR CRYOPRESERVATION

(71) Applicants: PanTHERA CryoSolutions Inc., Ottawa (CA); UNIVERSITY OF OTTAWA, Ottawa (CA)

(72) Inventors: Robert Ben, Ottawa (CA); André Paquette, Ottawa (CA)

(21) Appl. No.: 19/402,123

(22) Filed: Nov. 26, 2025

Related U.S. Application Data
(60) Provisional application No. 63/725,684, filed on Nov. 27, 2024.

(51) Int. Cl. C12N 5/00; C07C 273/18; C07C 275/10; C07C 275/26; C07C 275/30; C07D 213/40; C07D 295/215

(52) U.S. Cl. CPC C12N 5/525; C07C 273/1827; C07C 275/10; C07C 275/26; C07C 275/30; C07D 213/40; C07D 295/215; C12N 5/562

(57) ABSTRACT

The specification relates to a compound of formula (1), its enantiomer, diastereomer, or its physiologically acceptable salt thereof, along with a composition containing the same; wherein R¹, R², and R³ are as disclosed herein. The compound or composition have potential for use as a cryopreservation agent or an ice crystal recrystallization inhibitor. In addition, disclosed is a method for cryopreserving a biological material. Further, disclosed is a process for preparation of the compound.

### CROSS-REFERENCE TO RELATED APPLICATION

This application claims the benefit of U.S. Provisional Patent Application No. 63/725,684, filed Nov. 27, 2024, and incorporated by reference herein in its entirety.

### FIELD

The present specification pertains to the field of cryopreservation and sub-zero storage of biological material. In addition, the specification relates to a urea-based compound, a composition containing the urea compound, and use thereof. Furthermore, the specification relates to a urea compound as an ice recrystallization inhibitor, its method of manufacture and uses thereof in cryopreservation and sub-zero storage.

### BACKGROUND

Cryopreservation remains the most common method for the long-term storage of cells and can also be used for long-term storage of tissue. However, commonly during cryopreservation a significant number of cells experience irreparable damage due to the growth of ice, ultimately resulting in decreased post-thaw recoveries or impaired function. As cell-based therapeutics continue to define new models of care, for example, in stem cell therapy, regenerative medicine and transfusion, it is becoming increasingly important to ensure the highest level of post-thaw cell viability and functionality.

Currently, cryopreservation solutions include cryoprotective agents (CPAs), such as glycerol, dimethyl sulfoxide (DMSO) hydroxyethyl starch (HES) and others, which are used to mitigate cell damage during cryopreservation. However, cryopreservation solutions can include these CPAs at concentrations that can be toxic and damage the cells that they are meant to preserve.

Apart from the cytotoxicity of currently used CPAs, a significant short fall of most current cryopreservation solutions and protocols is their failure to control ice recrystallization. These CPAs do not protect against cell damage that ice crystals can cause when samples are frozen, stored, subjected to transient warming or during thawing. This cellular damage is a direct result of ice recrystallization and ultimately results in decreased post-thaw viability and functional capacity of the cells and tissues that have been cryopreserved. Ice recrystallization is a form of ice crystal re-modeling that occurs during freezing, maintenance at sub-zero temperatures and warming from sub-zero temperatures, and which results in the growth of large ice crystals at the expense of small ice crystals. Ice recrystallization significantly contributes to cell death from or during sub-zero storage of cells, tissues, and organs. This fact is evidenced by the membrane damage observed following cryopreservation of cells.

Stem cell and regenerative therapy using cryopreserved cells has been hampered by decreased cell function and viability after thawing. Consequently, improved cryopreservation protocols that increase the yield of viable and functional cells are urgently required. Improved cryopreservation compositions and methods have the potential to revolutionize cell and gene therapies.

Freeze-tolerant organisms have developed mechanisms to avoid the problems associated with ice recrystallization by producing large quantities of biological antifreeze proteins and glycoproteins that function, at least in part, as ice recrystallization inhibitors (IRls) in vivo. Unfortunately, biological antifreeze proteins and glycoproteins are typically not suitable for use as cryoprotectants in cryopreservation solutions because of their ice binding activity and/or dynamic ice shaping, which can exacerbate cellular damage. In addition, use of biological antifreeze proteins and glycoproteins is limited due to their poor solubility and the cost associated with isolation or manufacture.

Various polymers have been explored as an alternative to DMSO and glycerol, but thus far have failed to provide the high cell viabilities observed with DMSO or glycerol. Similarly, various sugars (mono- di- and oligosaccharides) have also been investigated as cryoprotectants. However, the structure of the carbohydrate, the freezing protocol, cell type and reported cell viabilities vary dramatically between studies making it difficult to ascertain the true ability of these compounds to protect cells against cryo-injury. More recently studies have pursued other small molecules that can function as IRIs. Following such studies, a particularly efficient ice recrystallization inhibitor, referred to as 2FA (depicted below), was identified to be useful in formulations for cryopreservation in cell and gene therapy applications.

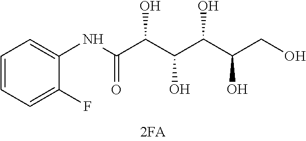 [SMILES: O=C(Nc1ccccc1F)[C@H](O)[C@@H](O)[C@H](O)[C@H](O)CO]

However, there is a need for an alternative compound and/or composition to overcome or mitigate at least some of the deficiencies of the prior art, or to provide a useful alternative. As such, there is a need for an alternative small molecule IRI, and for small molecule IRI that exhibits improved characteristics. An example of such characteristic can include improved solubility, while retaining the ability to control and/or reduce ice recrystallization.

The background herein is included solely to explain the context of the disclosure. This background is not to be taken as an admission that any of the material referred to was published, known, or part of the common general knowledge as of the priority date.

### SUMMARY

In one aspect, the specification relates to a compound of formula (I):

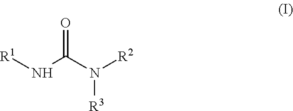

its enantiomer, diastereomer, or its physiologically acceptable salt thereof,
wherein
R¹ is

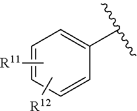

or a C₁₋₈ alkyl, optionally having one or more heteroatoms;
wherein each R¹¹ and R¹² independently is H, a C₁₋₄-alkyl optionally having one or more heteroatoms, a C₁₋₄-alkoxy optionally having one or more heteroatoms, —N(R^{a})₂, —C(═O)R^{b}, —CO₂R^{c}, —CHO or a halogen;
wherein each R^{a}, R^{b} and R^{c} independently is H, a C₁₋₄ alkyl optionally having one or more heteroatoms, or an aryl optionally having one or more heteroatoms;
R² and R³, together with the N atom to which they are bonded, form a three to eight membered ring, optionally having one or more heteroatoms in addition to the N atom to which R² and R³ are bonded, or
R³ is H, and R² is

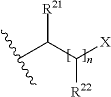

wherein
X is —OR^{d}, —N(R^{e})₂, —CO₂H, aryl, wherein R^{d} and each R^{e} independently is —H, a C₁₋₃-alkyl, or —CH₂—CH₂OH;
R²¹ is H, —OH, or —CH₂OH,
each R²² independently is H, —OH, —CH₂OH, or a C₁₋₃-alkyl optionally having one or more heteroatoms, with the proviso that when X is —OR^{d}, —N(R^{e})₂, aryl, R²² on the carbon atom attached to X is H; and
n is from 1 to 7.

In a second aspect, the specification relates to a cryopreservation agent comprising the compound of formula (I), as disclosed herein, its enantiomer, diastereomer, or its physiologically acceptable salt thereof.

In a third aspect, the specification relates to an ice crystal recrystallization inhibitor comprising the compound of formula (I), as disclosed herein, its enantiomer, diastereomer, or its physiologically acceptable salt thereof.

In a fourth aspect, the specification relates to a composition comprising the compound of formula (I), as disclosed herein, its enantiomer, diastereomer, or its physiologically acceptable salt thereof.

In a fifth aspect, the specification relates to a use of the compound of formula (I), as disclosed herein, its enantiomer, diastereomer, or its physiologically acceptable salt thereof, or the composition as disclosed herein, as a cryopreservation agent or ice crystal recrystallization inhibition in a biological material.

In a sixth aspect, the specification relates to a use of the compound of formula (I), as disclosed herein, its enantiomer, diastereomer, or its physiologically acceptable salt thereof, or the composition as disclosed herein, to:
a) reduce toxicity during cryopreservation in comparison to cryopreservation in the absence of the compound of formula (I), as disclosed herein, its enantiomer, diastereomer, or its physiologically acceptable salt thereof, or the composition as disclosed herein;
b) improve viability and/or functionality of biological material following cryopreservation in comparison to cryopreservation in the absence of the compound of formula (I), as disclosed herein, its enantiomer, diastereomer, or its physiologically acceptable salt thereof, or the composition as disclosed herein;
c) improve stability of biological material during temperature cycling in cryopreservation in comparison to temperature cycling in cryopreservation in the absence of the compound of formula (I), as disclosed herein, its enantiomer, diastereomer, or its physiologically acceptable salt thereof, or the composition as disclosed herein; and/or
d) facilitate cryopreservation at a warmer temperature than possible in the absence of the compound of formula (I), as disclosed herein, its enantiomer, diastereomer, or its physiologically acceptable salt thereof, or the composition as disclosed herein.

In a seventh aspect, the specification relates to a method for cryopreserving a biological material comprising:
a) combining the biological material with a solution comprising the compound of formula (I), as disclosed herein, its enantiomer, diastereomer, or its physiologically acceptable salt thereof, or the composition as disclosed herein;
b) cooling the combination from step a) to a storage temperature at or below the freezing point of the solution; and
c) storing the cooled combination at the storage temperature.

In an eighth aspect, the specification relates to a process for preparation of the compound of formula (I), as disclosed herein, its enantiomer, diastereomer, or its physiologically acceptable salt thereof, the process comprising the step of:
reacting an isocyanate of formula

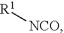

with an amine of formula

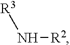

to form the compound of formula (I)

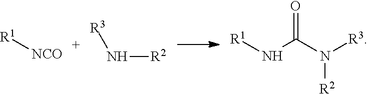

In a ninth aspect, the specification relates to a process for preparation of the compound of formula (I), as disclosed herein, its enantiomer, diastereomer, or its physiologically acceptable salt thereof, the process comprising the step of:
coupling a first amine of formula

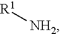

with a second amine of formula

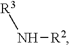

in the presence of a coupling agent to form the compound of formula (I)

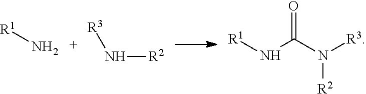

### DESCRIPTION OF EXAMPLE EMBODIMENTS

Unless otherwise explained, all technical and scientific terms used herein have the same meaning as commonly understood by one of ordinary skill in the art to which this disclosure belongs. Although any methods and materials similar or equivalent to those described herein can be used in the practice for testing of the present invention, the typical materials and methods are described herein. In describing and claiming the present invention, the common terminology generally used is described herein below. It is also to be understood that the terminology used herein is for the purpose of describing particular aspects only, and is not intended to be limiting.

Many patent applications, patents, and publications are referred to herein to assist in understanding the aspects described. Each of these references are incorporated herein by reference in their entirety.

When introducing elements disclosed herein, the articles “a”, “an”, “the”, and “said” are intended to mean that there may be one or more of the elements.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. It will be understood that any embodiments described as “comprising” certain components may also “consist of” or “consist essentially of,” these components, wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects described herein. For example, a composition defined using the phrase “consisting essentially of” encompasses any known acceptable additive, excipient, diluent, carrier, and the like, suitable for the composition described herein. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation, such as any specific compounds or method steps, whether implicitly or explicitly defined herein.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

The phrase “at least one of” is understood to be one or more. The phrase “at least one of . . . and . . . ” is understood to mean at least one of the elements listed or a combination thereof, if not explicitly listed. For example, “at least one of A, B, and C” is understood to mean A alone or B alone or C alone or a combination of A and B or a combination of A and C or a combination of B and C or a combination of A, B, and C.

The specification relates to a compound of formula (I), its enantiomer, diastereomer, or its physiologically acceptable salt thereof, along with a composition containing the same. The compound or composition have use as a cryopreservation agent or an ice crystal recrystallization inhibitor. In addition, disclosed is a method for cryopreserving a biological material using compound of formula (I), its enantiomer, diastereomer, or its physiologically acceptable salt thereof. Further, disclosed is a process for preparation of the compound of formula (I), its enantiomer, diastereomer, or its physiologically acceptable salt thereof.

The specification includes sub-heading to assist the reader with understanding the specification. The disclosure under one sub-heading is not limited to the sub-heading, rather the specification should be considered as a whole, with information under a particular sub-heading being considered and taken into account when reviewing another sub-heading, if applicable.

### The Compound

In one aspect, the specification relates to a compound of formula (I):

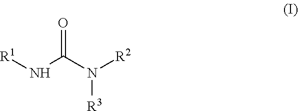

its enantiomer, diastereomer, or its physiologically acceptable salt thereof,
wherein
R¹ is

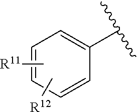

or a C₁₋₈ alkyl, optionally having one or more heteroatoms;
wherein each R¹¹ and R¹² independently is H, a C₁₋₄-alkyl optionally having one or more heteroatoms, a C₁₋₄-alkoxy optionally having one or more heteroatoms, —N(R^{a})₂, —C(═O)R^{b}, —CO₂R^{c}, —CHO or a halogen;
wherein each R^{a}, R^{b} and R^{c} independently is H, a C₁₋₄ alkyl optionally having one or more heteroatoms, or an aryl optionally having one or more heteroatoms;
R² and R³, together with the N atom to which they are bonded, form a three to eight membered ring, optionally having one or more heteroatoms in addition to the N atom to which R² and R³ are bonded, or
R³ is H, and R² is

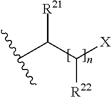

wherein
X is —OR^{d}, —N(R^{e})₂, —CO₂H, aryl, wherein R^{d} and each R^{e} independently is —H, a C₁₋₃-alkyl, or —CH₂—CH₂OH;
R²¹ is H, —OH, or —CH₂OH,
each R²² independently is H, —OH, —CH₂OH, or a C₁₋₃-alkyl optionally having one or more heteroatoms, with the proviso that when X is —OR^{d}, —N(R^{e})₂, or aryl, R²² on the carbon atom attached to X is H; and
n is from 1 to 7.

Certain structural features play a key role in the IRI activity of these urea-based small molecules, facilitating strong IRI activity, acceptable solubility in buffer and low levels of cytotoxicity. These molecules are often amphipathic, which can facilitate IRI activity and solubility in solution. The H-bond donors and acceptors conferred by the urea and the additional functional groups on the left side and the right side of the urea bond also tend to affect activity and solubility. Generally, the left side of the urea can be a substituted phenyl group or an alkyl chain while the right side of the urea can be a cyclic or linear alkyl group. The left side phenyl group requires substitution such as halogens, alkyl group or a variety of heteroatom-containing functional groups such as esters and amines for increased solubility and IRI activity. The left alkyl group containing molecules tend to have strong IRI activity, and when used at low or moderate concentrations, avoid potential solubility and cytotoxicity issues. The right side of the molecule can have a broader structure where cyclic groups containing heteroatoms can facilitate solubility and linear groups containing hydroxyls and amines play a key role in solubility and IRI activity.

The term, enantiomer, as used herein is not particularly limited and should be known or understood by a person of skill in the art. In organic chemistry, an enantiomer is a type of stereoisomer. Enantiomers, also known as optical isomers, are two stereoisomers that are related to each other by a reflection. Enantiomers are a pair of molecules that exist in two forms that are mirror images of one another but cannot be superimposed one upon the other. Enantiomers are in every other respect chemically identical.

The term, diastereomer, as used herein is not particularly limited and should be known or understood by a person of skill in the art. In organic chemistry, diastereomers are a type of stereoisomer. Diastereomers are defined as non-mirror image, non-identical stereoisomers. Hence, they occur when two or more stereoisomers of a compound have different configurations at one or more of the equivalent stereocenters and are not mirror images of each other.

The phrase, physiologically acceptable salt, as used herein is not particularly limited and should be known or understood by a person of skill in the art. A physiologically acceptable salt broadly refers to a salt of the compound of formula (I) that does not alter or have a detrimental effect on the normal functions of organisms or their parts. In addition, physiologically acceptable salts can include a salt that can help sustain the normal biological function of the organisms or their parts.

Non-limiting examples of such salts can include, without limitation, acid addition salts, formed with inorganic acids such as hydrochloric acid, hydrobromic acid, sulfuric acid, nitric acid, phosphoric acid, and the like; or formed with organic acids such as glycolic acid, pyruvic acid, lactic acid, malonic acid, malic acid, maleic acid, fumaric acid, tartaric acid, citric acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, lauryl sulfuric acid, gluconic acid, glutamic acid, salicylic acid, muconic acid, and the like; or basic addition salts formed with the conjugate bases of any of the inorganic acids listed above, wherein the conjugate bases comprise a cationic component selected from among Na⁺, K⁺, Mg⁺², Ca⁺², and quaternary ammonium. In one embodiment, for example and without limitation, where the compound of formula (I) has a positive charge, the counter-ion can be, for example and without limitation, HCO₃⁻, BF₄⁻, CO₃²⁻, NO₃⁻, ClO₄⁻, SO₄²⁻, F⁻, Br⁻, C₃H₃O²—, NH₃, MnO₄⁻, NO₂⁻, BrO₃⁻, IO₃⁻, Cr₂O₇²⁻, OH⁻, ClO³⁻, HCO²⁻, and the like.

The term, alkyl, as used herein is not particularly limited and should be known or understood by a person of skill in the art. An alkane as used herein is a saturated hydrocarbon. An alkyl group is an alkane missing one hydrogen. In any notation used herein, such as, for example and without limitation, C₁₋₃, C₁₋₄, or C₁₋₈, the subscript denotes the possible number of carbon atoms. For example, the term, C₁₋₃-alkyl as used herein refers to an alkyl group having from one to three carbon atoms. Non-limiting examples of alkyl include methyl, ethyl, propyl, isopropyl, butyl, iso-butyl, tert-butyl, pentyl, iso-pentyl, etc. The alkyl group may be a straight-chain, a branched-chain or cyclic. The term, alkyl, is intended to embrace all structural isomeric forms of an alkyl group. For example, as used herein, propyl encompasses both n-propyl and isopropyl; and butyl encompasses n-butyl, sec-butyl, isobutyl and tert-butyl.

The term, aryl, as used herein is not particularly limited and should be known or understood by a person of skill in the art. An aryl is any functional group or substituent derived from an aromatic ring, usually an aromatic hydrocarbon. In the subject application, an aryl group encompasses a heteroaryl, where the aryl group has a heteroatom as part of the aromatic ring. In addition, the term, aryl, as used herein, refers to a monocyclic or polycyclic aromatic group. Non-limiting examples of aryl group include phenyl, naphthyl, benzyl, pyridinyl, thienyl or indolyl. In one embodiment, for example and without limitation, aryl group has from three to ten carbon atoms. In another embodiment, for example and without limitation, the aryl group is a five or six membered ring.

The phrase, optionally having one or more heteroatoms, as used herein is not particularly limited and should be known or understood by a person of skill in the art. The phrase, having one or more heteroatoms, is used interchangeably herein with the term, heteroatom-modified. A heteroatom refers to any atom other than carbon and hydrogen. In one embodiment, for example and without limitation, the heteroatom is one or more of nitrogen, oxygen, sulphur or a halogen. The presence of the heteroatom can change the functional group of the organic substituent on which the heteroatom is present. For example, and without limitation, when an oxygen atom is present in an alkyl chain, the organic substituent can be an alcohol, an ether, an alkoxide, a ketone, or an aldehyde. The number of heteroatoms present on the organic substituent is not particularly limited and can be varied based on design and application requirements. In one embodiment, for example and without limitation, the organic substituent can have from one, two, three, four, five or six heteroatoms, and where the heteroatoms are the same or different.

The term, alkoxy, as used herein is not particularly limited and should be known or understood by a person of skill in the art. An alkoxy as used herein refers to straight-chain or branched alkyl group bonded to an oxygen. An alkoxy as used herein has the general formula R—O—, where R is an organic substituent. In any notation used herein, such as, for example and without limitation, C₁₋₃, C₁₋₄, or C₁₋₈, the subscript denotes the possible number of carbon atoms. For example, the term, C₁₋₃-alkoxy as used herein refers to an alkoxy group having from one to three carbon atoms. Non-limiting examples of alkoxy include methoxy, ethoxy, propoxy, butoxy, pentoxy, hexoxy, heptoxy and octoxy. The alkoxy group may be a straight-chain, a branched-chain or cyclic. The term, alkoxy, is intended to embrace all structural isomeric forms of an alkoxy group. For example, as used herein, propoxy encompasses both n-propoxy and isopropoxy, etc.

The term, halogen, as used herein is not particularly limited and should be known or understood by a person of skill in the art. The halogens are elements that form group 17 of the periodic table. Halogen include fluorine, chlorine, bromine, or iodine.

The phrase, three to eight membered ring, as used herein is not particularly limited and should be understood by a person of skill in the art. In the context of the specification, in the compound of formula (I), R² and R³, together with the N atom to which they are bonded, can form a three to eight membered ring. As such, the ring structure formed by R², R³ and the N atom to which R² and R³ are bonded can vary in size from a three membered ring to an eight membered ring. In one embodiment, for example and without limitation, R² and R³, together with the N atom to which they are bonded, form

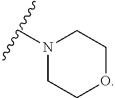 [SMILES: *N1CCOCC1]

In the compound of formula (I), when R³ is —H, R² is

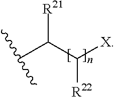

In such a structure, there are ‘n’ number of C units, with each unit having R²². The ‘n’ number of —CR²²— units form a linear carbon chain. The number of —CR²²— units can vary from 1 to 7, and are selected based on design and application requirements. Each R²² in —CR²²— units is independently selected from H, —OH, —CH₂OH, or a C₁₋₃-alkyl optionally having one or more heteroatoms. As such, for example and without limitation, if there are three units of —CR²²—, the three R²²'s present can be H, —OH, and —CH₂OH, or the three R²²'s present can all be H, —OH, or —CH₂OH. It should be noted that when X is —OR^{d}, —N(R^{e})₂, or aryl (Ar), R²² on the carbon atom attached to X is H. For example and without limitation, if there are three units of —CR²²—, the structure can be —CH(OH)—CH(OH)—CH₂—Ar, where the R²² in the underlined carbon (C) is —H.

Non-limiting examples of the compound of formula (I) include:

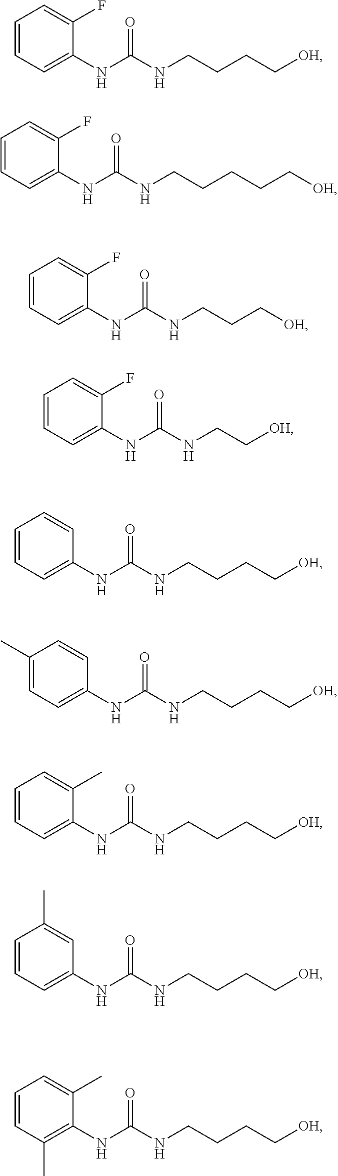

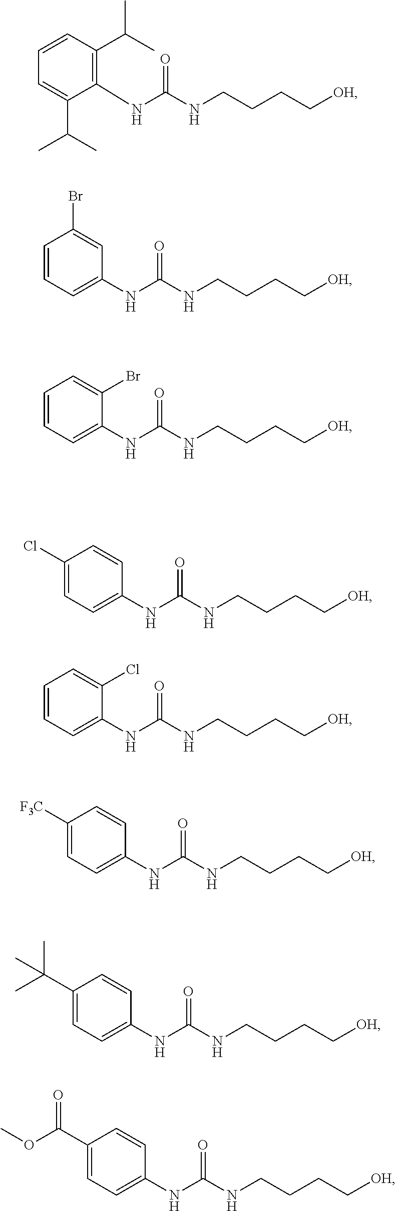

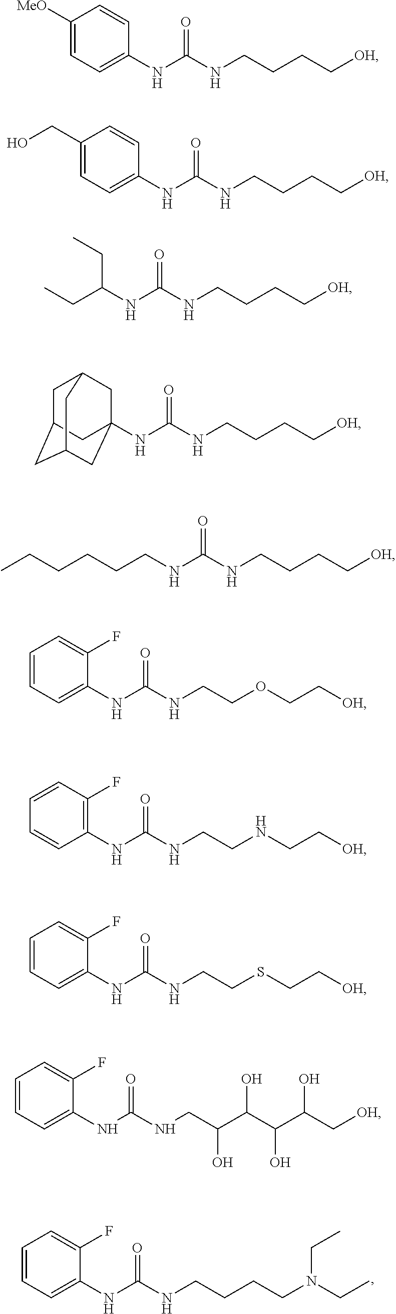

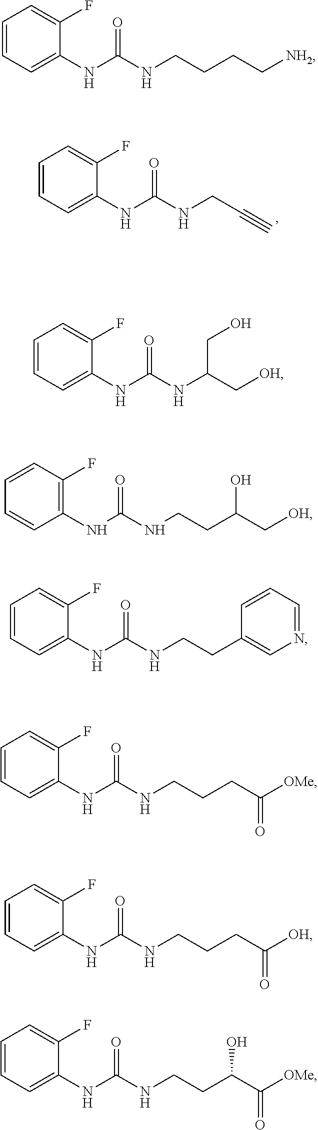

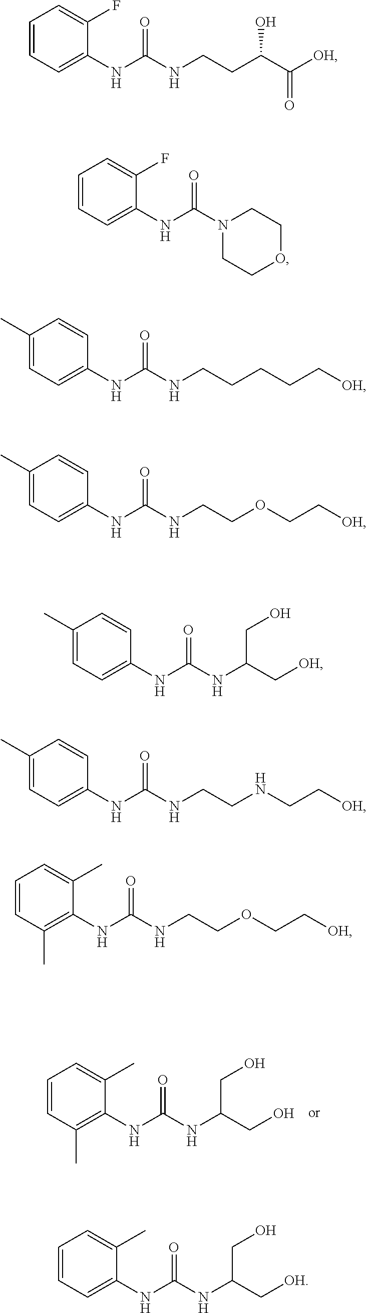

In a second aspect, the specification relates to a cryopreservation agent comprising the compound of formula (I), as disclosed herein, its enantiomer, diastereomer, or its physiologically acceptable salt thereof.

The term, cryopreservation agent or cryoprotective agent, as used herein is not particularly limited, and should be known or understood by a person of skill in the art. Cryoprotective agents (CPAs) are substances that protect cells or biological samples when frozen. In one embodiment, for example and without limitation, CPA's can include substances that prevent or reduce the formation of ice crystals when cells or biological are frozen. CPAs can be classified as permeating or non-permeating based on their molecular weight. Non-limiting examples of CPA's include dimethyl sulfoxide (DMSO), glycerol, trehalose, ethylene glycol (EG) or propylene glycol (PG).

In a third aspect, the specification relates to an ice crystal recrystallization inhibitor comprising the compound of formula (I), as disclosed herein, its enantiomer, diastereomer, or its physiologically acceptable salt thereof.

The term, ice crystal recrystallization inhibitor (IRI), as used herein is not particularly limited, and should be known or understood by a person of skill in the art. Ice recrystallization inhibitors (IRI's) are substances that can prevent ice crystals from growing too large in a frozen solution. They are used as cryoprotective agents to reduce damage to cells, tissues, and organs during cryopreservation. A non-limiting example of an IRI is 2FA (disclosed herein).

### Composition

In a fourth aspect, the specification relates to a composition comprising the compound of formula (I), as disclosed herein, its enantiomer, diastereomer, or its physiologically acceptable salt thereof.

Also provided herein are compositions for preserving biological material comprising one or more of the compounds of Formula (I), optionally in combination with one or more cryoprotecting agents and/or ice recrystallization inhibitor, as disclosed herein. The preservation compositions disclosed herein are suitable for use in cryopreservation and/or ice recrystallization inhibitor, in which the compositions comprising the biological material are frozen, or for subzero preservation without freezing. In both instances, the presence of one or more compounds disclosed herein aids in inhibiting ice recrystallization formation during cooling and during warming following cryopreservation. The preservation compositions of the present application can also be used in non-cryopreservation storage methods, such as, for example and without limitation, hypothermic/vitrification storage methods and normothermic storage methods.

In an embodiment, for example and without limitation, the at least one compound of formula (I), disclosed herein, is present in the composition at a concentration of less than about 400 mM, less than about 200 mM, less than about 100 mM, less than about 10 mM, less than about 1 mM, or less than about 0.5 mM. In another embodiment, for example and without limitation, the compound of formula (1), disclosed herein, can be present in the composition at a concentration of about 0.5 mM to (and including) about 400 mM, or about 55 mM to (and including) about 220 mM.

As should be understood by the skilled person, the amount of compound of formula (I), disclosed herein, employed in a composition will be determined, in part, based on the ratio between the toxicity of the compound its IRI and/or CPA activity. This determination method is analogous to a therapeutic index as employed in determining appropriate dosing of a drug. In accordance with the specification, the concentration of the compound of formula (I), disclosed herein, used in the composition will be determined, in part, based on the ratio of the LD₅₀ and the IC₅₀ of the compound of formula (I), disclosed herein, with a higher ratio indicative of the ability to use a higher concentration of the compound of formula (I), disclosed herein, while minimizing or avoiding cytotoxicity during cryopreservation or ice recrystallization inhibition.

In an embodiment in accordance with the specification, the compound or compositions disclosed herein can be used for cryopreserving a biological material, wherein the composition additionally comprises at least one, at least two, or at least three, cryoprotecting agents. The cryoprotecting agent selected is not particularly limited and can vary depending upon design and application requirements. In one embodiment, for example and without limitation, the cryoprotecting agent is dimethyl sulfoxide (DMSO), lactobionate, glycerol, trehalose, mannitol, glutathione, sucrose, hydroxyethyl starch (HES), ethylene glycol (EG), propylene glycol (PG), polyvinyl alcohol and/or other biopolymers useful in cryopreservation. The at least one cryoprotecting agent is present in the composition in a concentration of about 0.1 wt % to (and including) about 30 wt %, about 0.1 wt % to (and including) about 20 wt %, about 5 wt % to (and including) about 30 wt %, or about 5 wt % to (and including) about 20 wt %.

### Use and Methods

In a fifth aspect, the specification relates to a use of the compound of formula (I), as disclosed herein, its enantiomer, diastereomer, or its physiologically acceptable salt thereof, or the composition as disclosed herein, as a cryopreservation agent or ice crystal recrystallization inhibition in a biological material.

In an embodiment in accordance with the specification, the compound or compound composition, disclosed herein are used as a cryopreservation agent or for ice crystal recrystallization inhibition in a biological material. The term, biological material, as used herein is not particularly limited and should be known or understood by a person of skill in the art. Biological material can be, for example and without limitation, organs, tissues (e.g., organoids), food, cells, platelets, eggs, and embryos. Alternatively, the biological material can be cultured cells, tissues or organs. Examples of “cells” include, but are not limited to, stem cells, T cells (e.g., CAR-T cells), oocytes, sperm, peripheral blood mononuclear cells (PMBCs), neurons, progenitor cells, liver cells, red blood cells, immune cells (e.g., NK cells), endothelial cells, pancreatic cells (e.g., pancreatic islet cells), dendritic cells, fibroblasts or cells from a cell line. The cells can be in an isolated or a purified form, or they can be in a mixture with one or more other cell types.

In some embodiments, the biological material is from a non-human animal or human source. For example, the biological material from a non-human animal or human source can be a cell, tissue or organ. In alternative embodiments, the biological material is from a plant source. For example, the plant-sourced biological material can be a plant cell, tissue or organ or can be plant seed or fruit, or a part thereof. In other embodiments, the biological material is from a fungal source. In yet other embodiments, the biological material comprises microorganisms of one or more types or species.

In an embodiment in accordance with the specification, the composition for cryopreserving or ice recrystallization inhibition of a biological material, as disclosed herein, further comprise a cell culture or growth medium. Examples of suitable cell culture media include, but are not limited to, Eagle's Minimum Essential Medium (MEM), Dulbecco's Modified Eagle's Medium (DMEM), Roswell Park Memorial Institute medium (RPMI), Fetal Bovine Serum (FBS), Fetal Calf Serum (FCS), Ham's F-10, Ham's F-12, Hank's buffered salt solution (HBSS), HBSS and dextrose, Medium 199 and combinations thereof.

In another embodiment, the composition for cryopreservation or ice recrystallization inhibition can contain both biological material and cell medium as provided above.

In a sixth aspect, the specification relates to a use of the compound of formula (I), as disclosed herein, its enantiomer, diastereomer, or its physiologically acceptable salt thereof, or the composition as disclosed herein, to:
a) reduce toxicity during cryopreservation in comparison to cryopreservation in the absence of the compound of formula (I), as disclosed herein, its enantiomer, diastereomer, or its physiologically acceptable salt thereof, or the composition as disclosed herein;
b) improve viability and/or functionality of biological material following cryopreservation in comparison to cryopreservation in the absence of the compound of formula (I), as disclosed herein, its enantiomer, diastereomer, or its physiologically acceptable salt thereof, or the composition as disclosed herein;
c) improve stability of biological material during temperature cycling in cryopreservation in comparison to temperature cycling in cryopreservation in the absence of the compound of formula (I), as disclosed herein, its enantiomer, diastereomer, or its physiologically acceptable salt thereof, or the composition as disclosed herein; and/or
d) facilitate cryopreservation at a warmer temperature than possible in the absence of the compound of formula (I), as disclosed herein, its enantiomer, diastereomer, or its physiologically acceptable salt thereof, or the composition as disclosed herein.

In a seventh aspect, the specification relates to a method for cryopreserving a biological material comprising:
a) combining the biological material with a solution comprising the compound of formula (I), as disclosed herein, its enantiomer, diastereomer, or its physiologically acceptable salt thereof, or the composition as disclosed herein;
b) cooling the combination from step a) to a storage temperature at or below the freezing point of the solution; and
c) storing the cooled combination at the storage temperature.

The use and method for cryopreserving or ice recrystallization inhibition of a biological material are provided herein in which the biological material is combined with in a solution of at least a compound of Formula (I), or a composition containing the compound of formula (I), and the resulting combination is frozen or cooled to a sub-zero storage temperature. As should be readily understood by the skilled person, the technique by which the biological material is combined with a solution of the at least one compound of Formula (I), or a composition containing the compound of formula (I), will be dependent on the nature of the biological material. For example, cells may be suspended in the solution prior to storage, or an organ may be perfused with the solution using the existing vasculature of the organ, or a tissue may be perfused with or immersed in the solution. Selection of the appropriate technique for combining the biomaterial with the solution is a matter of routine for the skilled person having knowledge of cryopreservation.

Accordingly, in some embodiments, use or method for cryopreserving or ice recrystallization inhibition of a biological material, in which the biological material is cells, are provided herein which comprise suspending the biological material in a solution of at least one compound of Formula (I), or a composition containing the compound of formula (I), to form a suspension and freezing the suspension or cooling the suspension to a sub-zero storage temperature.

In some embodiments, the use or method comprises adding a biological material to a solution comprising at least one compound of Formula (I), or a composition containing the compound of formula (I), and then cryopreserving the biological material in cryogenic vials or other suitable container. The vials can be frozen under rate controlled freezing conditions, such as freezing at 1° C. per minute over 16 hours. The vials can be stored using standard cryopreservation techniques, and then they can be thawed when required by removing the vials from the cold storage, and thawing using standard protocols. Examples of standard cryopreservation techniques include freezing in liquid nitrogen to about −196° C. and freezing in dry ice to about −80° C. Examples of standard thawing protocols include, but are not limited to, ambient thaw or rapid thaw in a water bath at a temperature that is at or between room temperature and 37° C. Alternatively, a controlled, slow thaw protocol can be used.

In a particular embodiment, for example and without limitation, there is provided a use or method for cryopreserving or ice recrystallization inhibition of a biological material by suspending biological material in a solution of at least one compound of Formula (I), or a composition containing the compound of formula (I), to form a suspension, which is contained in a vial or other suitable container, wherein the vial is frozen directly in a storage unit without a rate controlled freezing protocol.

Also provided herein is a use or method for inhibiting ice recrystallization during cryopreservation or sub-zero storage of a biological material, which method comprises combining, such as, by suspending, a biological material in a solution of at least one compound of Formula (I), or a composition containing the compound of formula (I), disclosed herein, to form a combination (e.g., a suspension) and cryopreserving the resultant suspension or cooling the resultant suspension to a sub-zero storage temperature.

Table 1 shows examples of the compound of formula (I) and their IC₅₀ and LD₅₀ values.

| Compound | IC₅₀ (mM) | LD₅₀ (mM) |
|---|---|---|
| 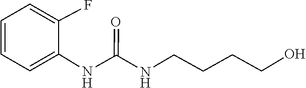 [SMILES: O=C(NCCCCO)Nc1ccccc1F] | 1 | 5 |
| 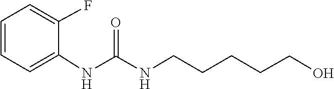 [SMILES: O=C(NCCCCCO)Nc1ccccc1F] | <10 | |
| 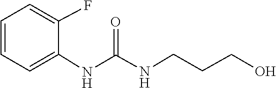 [SMILES: O=C(NCCCO)Nc1ccccc1F] | >10 | |
| 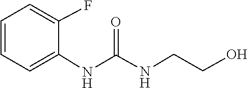 [SMILES: O=C(NCCO)Nc1ccccc1F] | >10 | |
| 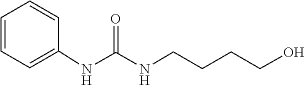 [SMILES: O=C(NCCCCO)Nc1ccccc1] | >10 | |
| 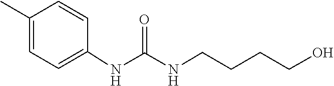 [SMILES: Cc1ccc(NC(=O)NCCCCO)cc1] | <1 | |
| 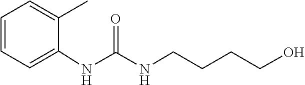 [SMILES: Cc1ccccc1NC(=O)NCCCCO] | <10 | |
| 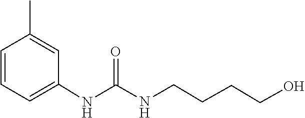 [SMILES: Cc1cccc(NC(=O)NCCCCO)c1] | <10 | |
| 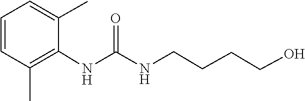 [SMILES: Cc1cccc(C)c1NC(=O)NCCCCO] | <10 | |
| 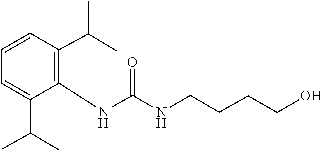 [SMILES: CC(C)c1cccc(C(C)C)c1NC(=O)NCCCCO] | | |
| 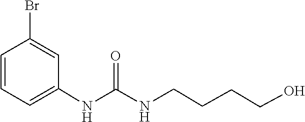 [SMILES: O=C(NCCCCO)Nc1cccc(Br)c1] | | |
| 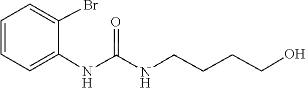 [SMILES: O=C(NCCCCO)Nc1ccccc1Br] | | |
| 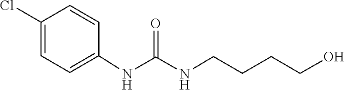 [SMILES: O=C(NCCCCO)Nc1ccc(Cl)cc1] | | |
| 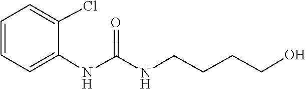 [SMILES: O=C(NCCCCO)Nc1ccccc1Cl] | | |
| 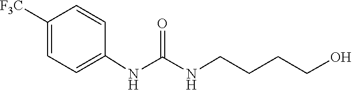 [SMILES: O=C(NCCCCO)Nc1ccc(C(F)(F)F)cc1] | | |
| 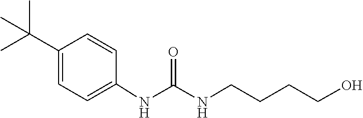 [SMILES: CC(C)(C)c1ccc(NC(=O)NCCCCO)cc1] | | |
| 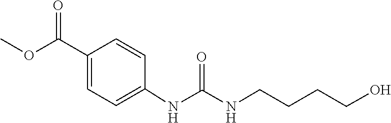 [SMILES: COC(=O)c1ccc(NC(=O)NCCCCO)cc1] | | |
| 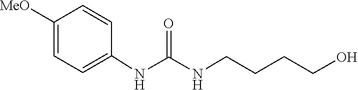 [SMILES: COc1ccc(NC(=O)NCCCCO)cc1] | <10 | |
| 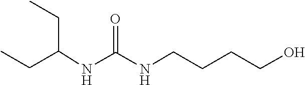 | >10 | |
| 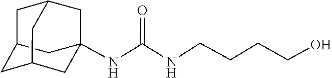 [SMILES: O=C(NCCCCO)NC12CC3CC(CC(C3)C1)C2] | | |
| 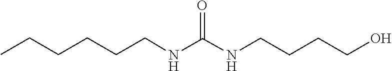 | <10 | |
| 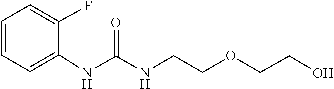 [SMILES: O=C(NCCOCCO)Nc1ccccc1F] | >10 | |
| 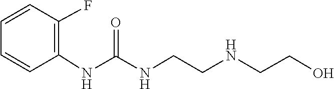 [SMILES: O=C(NCCNCCO)Nc1ccccc1F] | TBD | |
| 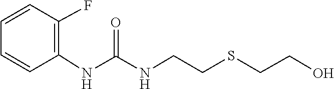 [SMILES: O=C(NCCSCCO)Nc1ccccc1F] | | |
| 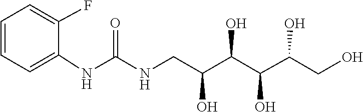 [SMILES: O=C(NC[C@H](O)[C@@H](O)[C@H](O)[C@H](O)CO)Nc1ccccc1F] | >10 | |
| 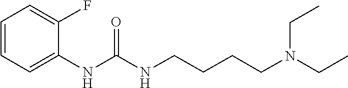 [SMILES: CCN(CC)CCCCNC(=O)Nc1ccccc1F] | >10 | |
| 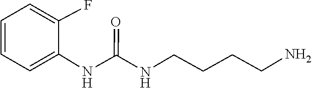 [SMILES: NCCCCNC(=O)Nc1ccccc1F] | >10 | |
| 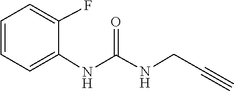 | | |
| 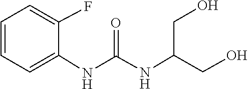 [SMILES: O=C(Nc1ccccc1F)NC(CO)CO] | ~0.33 | 6 |
| 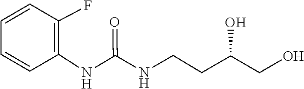 [SMILES: O=C(NCC[C@H](O)CO)Nc1ccccc1F] | <10 | 7.8 |
| 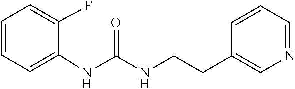 [SMILES: O=C(NCCc1cccnc1)Nc1ccccc1F] | >10 | |
| 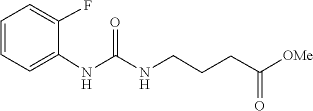 [SMILES: COC(=O)CCCNC(=O)Nc1ccccc1F] | | |
| 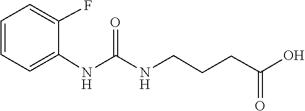 [SMILES: O=C(O)CCCNC(=O)Nc1ccccc1F] | ~10 | |
| 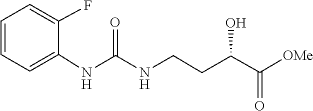 [SMILES: COC(=O)[C@@H](O)CCNC(=O)Nc1ccccc1F] | | |
| 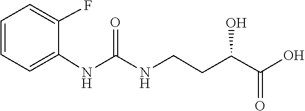 [SMILES: O=C(NCC[C@H](O)C(=O)O)Nc1ccccc1F] | >10 | |
| 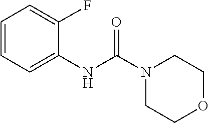 [SMILES: O=C(Nc1ccccc1F)N1CCOCC1] | >10 | |
| 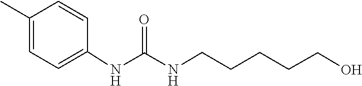 [SMILES: Cc1ccc(NC(=O)NCCCCCO)cc1] | <10 | |
| 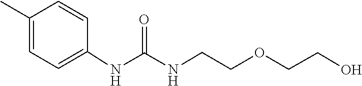 [SMILES: Cc1ccc(NC(=O)NCCOCCO)cc1] | 1 | 3.5 |
| 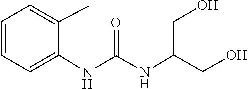 [SMILES: Cc1ccccc1NC(=O)NC(CO)CO] | ~0.25 | 2.6 |
| 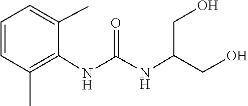 [SMILES: Cc1cccc(C)c1NC(=O)NC(CO)CO] | 0.180 | 2.5 |
| 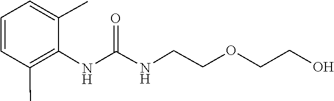 [SMILES: Cc1cccc(C)c1NC(=O)NCCOCCO] | ~0.2 | 3.8 |

### Kits for Preserving Biological Material

Kits for cryopreserving a biological material are provided herein comprising a compound of Formula (I), or a composition containing the compound of formula (I), disclosed herein, for cryopreserving or ice recrystallization inhibition of a biological material as disclosed herein. The compound and a further cryoprotecting agent, if present, can be in the same composition or in separate compositions. Additionally, they can be co-packaged for common presentation or packaged individually. Instructions can also be provided in the kit for cryopreservation or ice recrystallization inhibition of various types of biological material. The kits provided herein can further comprise a cell culture or growth medium.

### Process of Preparation

In an eighth aspect, the specification relates to a process for preparation of the compound of formula (I), as disclosed herein, its enantiomer, diastereomer, or its physiologically acceptable salt thereof, the process comprising the step of:
reacting an isocyanate of formula

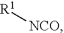

with an amine of formula

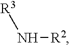

to form the compound of formula (I)

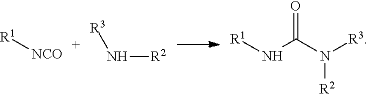

The process for preparation of the compound of formula (I) is not particularly limited. In one embodiment, an isocyanate is reacted with an amine in an appropriate solvent and reaction conditions to form the compound of formula (I). The R groups present on the isocyanate or amine are not particularly limited, and will depend on the compound of formula (I) being formed. Amine can function as a nucleophilic and add to the isocyanate that has an electrophilic carbon to form the compound of formula (I). The solvent used for carrying out the reaction should avoid participating in the reaction. As such, the solvent should be an aprotic solvent. The aprotic solvent used is not particularly limited. In one embodiment, for example and without limitation, the aprotic solvent is acetonitrile (ACN) or dichloromethane (DCM).

In an embodiment, for example and without limitation, a first amine can be converted into an isocyanate, followed by reacting of the isocyanate with an amine, as described herein. The conversion of the amine to the isocyanate is not particularly limited, and should be known or can be determined by a person of skill in the art (Knõlker, H.-J., et al., Angew. Chem., 1995, 107, 22, 2746, incorporated herein by reference). A non-limiting example of conversion of an amine to an isocyanate is shown below:

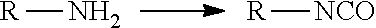

In a ninth aspect, the specification relates to a process for preparation of the compound of formula (I), as disclosed herein, its enantiomer, diastereomer, or its physiologically acceptable salt thereof, the process comprising the step of:
coupling a first amine of formula

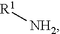

with a second amine of formula

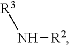

in the presence of a coupling agent to form the compound of formula (I)

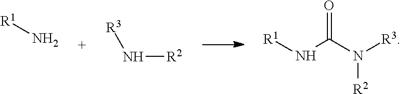

In another embodiment in accordance with the specification, two amine compounds can be coupled using a coupling agent to form the compound of formula (I). The coupling agent used is not particularly limited and should be known to a person of skill in the art. In one embodiment, for example and without limitation, the coupling agent is carbonyldiimidazole (CDI), or triphosgene.

### EXAMPLES

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Without further description, it is believed that one of ordinary skill in the art can, using the preceding description and the following illustrative examples, make and utilize the constructs of the present invention and practice the claimed methods. The following working examples therefore, specifically point out the typical aspects of the present invention and are not to be construed as limiting in any way in the remainder of the disclosure. Although specific terms have been employed herein, such terms are intended in a descriptive sense and not for purposes of limitation.

The synthesis of the compounds was carried out using two general procedures (General Procedure 1 or General Procedure 2), as noted below. The general procedure utilized in the synthesis of the compounds is noted under the compound synthesized. Using these two general procedures, a number of compounds were synthesized, as noted below. All starting materials and reagents were commercially available.

### List of Abbreviations

ACN—Acetonitrile
THF—Tetrahydrofuran
DCM—Dichloromethane or methylene chloride
CDI—N,N′-Carbonyldiimidazole
DIEA—N,N-Diisopropylethylamine
EtOAc—Ethyl acetate
EtOH—Ethanol
TsCl—4-Toluenesulfonyl chloride
Et₃N—Triethylamine
DMAP—4—Dimethylaminopyridine
DMF—N,N′—Dimethylformamide
MecOH—Methanol
TFA—Trifluoroacetic acid
BTC—Triphosgene

### Example 1: General Procedure 1

Isocyanate (1 equiv) was dissolved in ACN (0.1 M) and amine (1.1 equiv) was added dropwise. The solution was stirred at room temperature for 2 h, with a white precipitate forming. The solution was filtered over vacuum, washed with Hexanes and dried for 30 mins. The powder was collected, affording pure product (80-95% yield)

### Example 2: General Procedure 2

Isocyanate (1 equiv) was dissolved in ACN (0.2 M) and amine (1.1 equiv) was added dropwise. The solution was stirred at room temperature for 2 h and the solvent was removed under reduced pressure. The residue was purified by flash column chromatography to afford purified urea product (80-99% yield).

### Example 3: Compound 1

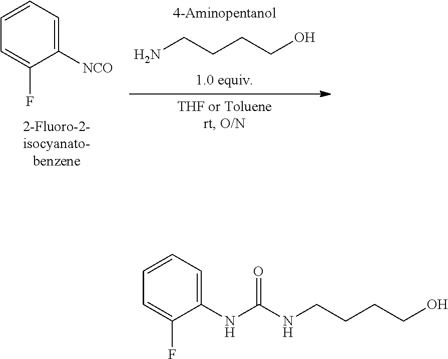

General procedure 1: 2-Fluorophenyl isocyanate (163 μL, 1.46 mmol, 1 equiv.), ACN (30 mL, 0.05 M) and 5-Aminopentanol (166 mg, 1.6 mmol, 1.1 equiv.). White powder.

¹H NMR (400 MHz, DMSO): δ 8.24 (d, J=2.7 Hz, 1H), 8.12 (td, J=8.3, 1.7 Hz, 1H), 7.16 (ddd, J=11.8, 8.1, 1.5 Hz, 1H), 7.10-7.02 (m, 1H), 6.95-6.86 (m, 1H), 6.60 (t, J=5.6 Hz, 1H), 4.43 (t, J=5.1 Hz, 1H), 3.41 (qd, J=5.4, 3.1 Hz, 2H), 3.09 (qd, J=5.5, 3.7 Hz, 2H), 1.50-1.39 (m, 4H).

¹³C NMR (151 MHz, DMSO): δ 154.8, 151.5 (d, J=240.4 Hz), 128.4 (d, J=10.1 Hz), 124.3 (d, J=3.3 Hz), 121.3 (d, J=7.3 Hz), 120.0, 114.7 (d, J=19.0 Hz), 60.4, 38.9, 29.9, 26.3.

### Example 4: Compound 2

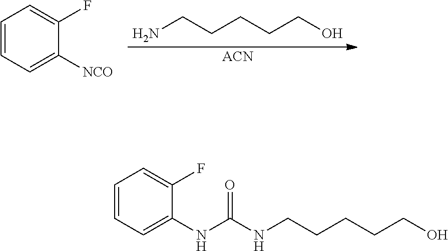

General procedure 2: 2-Fluorophenyl isocyanate (163 μL, 1.46 mmol, 1 equiv.), ACN (30 mL, 0.05 M) and 5-Aminopentanol (166 mg, 1.6 mmol, 1.1 equiv.). Flash column chromatography (80% EtOAc in Hexanes), white powder.

¹H NMR (600 MHz, DMSO): δ 8.24 (d, J=2.8 Hz, 1H), 8.14 (t, J=8.3 Hz, 1H), 7.14 (ddd, J=11.9, 8.1, 1.5 Hz, 1H), 7.05 (t, J=7.9 Hz, 1H), 6.89 (td, J=7.6, 5.4 Hz, 1H), 6.61 (t, J=5.7 Hz, 1H), 4.41-4.36 (m, 1H), 3.43-3.37 (m, 3H), 3.09 (q, J=6.5 Hz, 2H), 1.43 (h, J=6.8 Hz, 4H), 1.32 (qd, J=9.1, 5.9 Hz, 2H).

¹³C NMR (151 MHz, DMSO): δ 154.9, 151.6 (d, J=240.3 Hz), 128.5 (d, J=10.2 Hz), 124.4 (d, J=3.4 Hz), 121.3 (d, J=7.3 Hz), 120.1, 114.7 (d, J=18.9 Hz), 60.7, 39.1, 32.3, 29.6, 23.0.

### Example 5: Compound 3

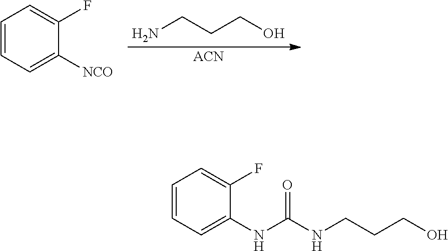

General procedure 2: 2-Fluorophenyl isocyanate (50 μL, 0.44 mmol, 1 equiv.), ACN (8.8 mL, 0.05 M) and 3-Aminopropanol (38 μL, 0.49 mmol, 1.1 equiv.). Flash column chromatography (50-75% EtOAc in Hexanes), white powder.

¹H NMR (600 MHz, DMSO): δ 8.25 (d, J=2.7 Hz, 1H), 8.10 (tdd, J=8.3, 3.8, 1.6 Hz, 1H), 7.15 (ddd, J=11.7, 8.2, 1.4 Hz, 1H), 7.06 (td, J=8.0, 1.6 Hz, 1H), 6.95-6.86 (m, 1H), 6.60 (q, J=5.9 Hz, 1H), 3.48-3.42 (m, 2H), 3.14 (td, J=6.8, 5.4 Hz, 2H), 1.57 (p, J=6.6 Hz, 2H).

¹³C NMR (151 MHz, DMSO): δ 154.9, 151.5 (d, J=240.4 Hz), 128.2 (d, J=10.2 Hz), 124.23 (d, J=3.2 Hz), 121.3 (d, J=7.1 Hz), 120.1, 114.6 (d, J=19.0 Hz), 58.1, 36.1, 32.6.

### Example 6: Compound 4

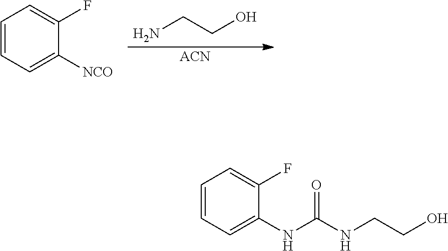

General procedure 2: 2-Fluorophenyl isocyanate (50 μL, 0.44 mmol, 1 equiv.), ACN (8.8 mL, 0.05 M) and ethanolamine (29 μL, 0.48 mmol, 1.1 equiv.). Flash column chromatography (50-75% EtOAc in Hexanes), white powder.

¹H NMR (600 MHz, DMSO): δ 8.37 (d, J=2.7 Hz, 1H), 8.11 (td, J=8.3, 1.7 Hz, 1H), 7.15 (ddd, J=11.8, 8.2, 1.4 Hz, 1H), 7.09-7.03 (m, 1H), 6.90 (tdd, J=7.3, 5.1, 1.6 Hz, 1H), 6.73 (t, J=5.6 Hz, 1H), 4.74 (t, J=5.1 Hz, 1H), 3.44 (q, J=5.4 Hz, 2H), 3.16 (q, J=5.6 Hz, 2H).

¹³C NMR (151 MHz, DMSO): δ 155.0, 151.6 (d, J=240.5 Hz), 128.4 (d, J=10.2 Hz), 124.3 (d, J=3.3 Hz), 121.4 (d, J=7.4 Hz), 120.2, 114.7 (d, J=19.0 Hz), 60.3, 41.8.

### Example 7: Compound 5

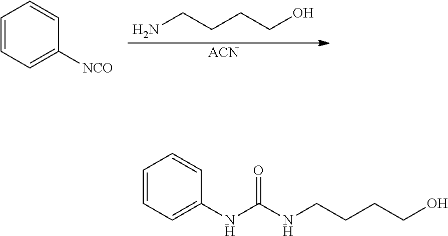

General procedure 1: Phenyl isocyanate (160 μL, 1.47 mmol, 1 equiv.), ACN (20 mL, 0.05 M) and morpholine (135 μL, 1.33 mmol, 1 equiv.). White powder.

¹H NMR (400 MHz, DMSO): δ 8.41 (s, 1H), 7.41-7.32 (m, 1H), 7.20 (dd, J=8.5, 7.3 Hz, 2H), 6.91-6.83 (m, 1H), 6.12 (d, J=5.6 Hz, 1H), 4.46 (t, J=5.1 Hz, 1H), 3.14-3.00 (m, 2H), 1.43 (td, J=5.4, 3.2 Hz, 4H).

¹³C NMR (101 MHz, DMSO): δ 155.3, 140.7, 128.7, 121.0, 117.6, 60.6, 40.1, 39.0, 29.9, 26.5.

### Example 8: Compound 6

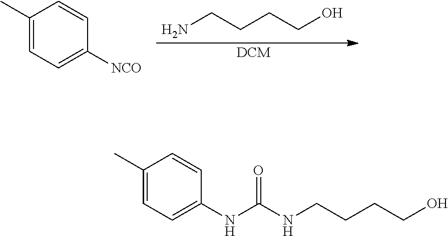

General procedure 1: 4-Methyl phenylisocyanate (57 μL, 0.45 mmol, 1 equiv.), DCM (2.2 mL, 0.2 M) and 4-aminobutanol (41.1 μL, 0.45 mmol, 1 equiv.). White powder.

¹H NMR (400 MHz, DMSO): δ 8.26 (s, 1H), 7.25 (d, J=8.4 Hz, 2H), 7.00 (d, J=8.2 Hz, 2H), 6.04 (t, J=5.7 Hz, 1H), 4.42 (t, J=5.1 Hz, 1H), 3.40 (td, J=6.4, 4.0 Hz, 2H), 3.06 (dq, J=7.3, 4.2 Hz, 2H), 2.20 (s, 3H), 1.43 (p, J=3.3 Hz, 4H).

¹³C NMR (151 MHz, DMSO): δ 155.3, 138.0, 129.6, 129.0, 117.7, 60.5, 38.9, 29.9, 26.5, 20.3.

### Example 9: Compound 7

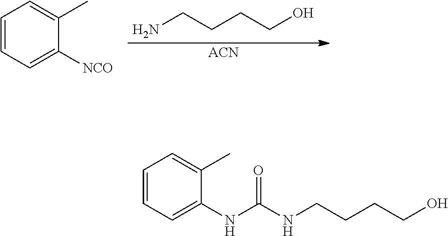

General procedure 1: 2-Methylphenyl isocyanate (134 μL, 1.00 mmol, 1 equiv.), ACN (10 mL, 0.1 M) and 4-aminobutanol (92 μL, 1.00 mmol, 1 equiv.). White powder

¹H NMR (600 MHz, DMSO): δ 7.81 (dd, J=8.2, 1.3 Hz, 1H), 7.56 (s, 1H), 7.10 (dd, J=7.3, 1.7 Hz, 1H), 7.07 (ddd, J=8.9, 7.5, 1.6 Hz, 1H), 6.85 (td, J=7.4, 1.3 Hz, 1H), 6.51 (t, J=5.6 Hz, 1H), 4.42 (t, J=5.1 Hz, 1H), 3.42 (dt, J=7.7, 5.4 Hz, 2H), 3.11-3.05 (m, 2H), 2.17 (s, 3H), 1.45 (p, J=3.4 Hz, 4H).

¹³C NMR (151 MHz, DMSO): δ 155.4, 138.3, 130.0, 126.5, 126.0, 121.7, 120.3, 60.5, 39.0, 29.9, 26.4, 17.9.

### Example 10: Compound 8

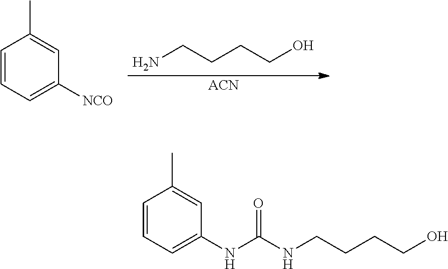

General procedure 1: 3-Methylphenyl isocyanate (100 μL, 0.81 mmol, 1 equiv.), ACN (8 mL, 0.1 M) and 4-aminobutanol (74 μL, 0.81 mmol, 1 equiv.). White powder

¹H NMR (600 MHz, DMSO): δ 8.28 (s, 1H), 7.21 (d, J=2.4 Hz, 1H), 7.15 (d, J=8.2 Hz, 1H), 7.10-7.05 (m, 1H), 6.69 (d, J=7.4 Hz, 1H), 6.08 (t, J=5.7 Hz, 1H), 4.42 (t, J=5.1 Hz, 1H), 3.41 (q, J=5.7 Hz, 2H), 3.10-3.04 (m, 2H), 2.23 (s, 3H), 1.44 (p, J=3.6 Hz, 4H).

¹³C NMR (151 MHz, DMSO): δ 155.2, 140.5, 137.7, 128.5, 121.7, 118.1, 114.8, 60.5, 39.0, 29.9, 26.5, 21.3.

### Example 11: Compound 9

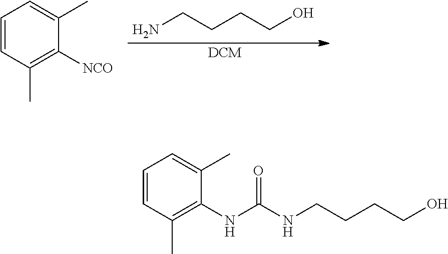

General procedure 1: 2,6-Dimethylphenyl isocyanate (162 μL, 1.16 mmol, 1 equiv.), ACN (5.8 mL, 0.2 M) and 4-aminobutanol (100 μL, 1.16 mmol, 1 equiv.). White powder.

¹H NMR (600 MHz, DMSO): δ 7.37 (s, 1H), 7.01 (q, J=5.3 Hz, 3H), 5.98 (s, 1H), 4.39 (t, J=5.1 Hz, 1H), 3.40 (d, J=5.3 Hz, 2H), 3.04 (t, J=6.2 Hz, 2H), 2.14 (s, 6H), 1.43 (p, J=3.2 Hz, 4H).

¹³C NMR (151 MHz, DMSO): δ 156.0, 136.2, 135.6, 127.6, 125.5, 60.5, 40.1, 29.8, 26.7, 18.2.

### Example 12: Compound 10

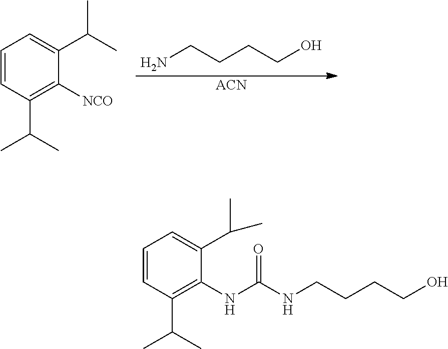

General procedure 1: 2,6-Diisopropylphenyl isocyanate (214 μL, 1.00 mmol, 1 equiv.), ACN (10 mL, 0.1 M) and 4-aminobutanol (92 μL, 1.00 mmol, 1 equiv.). White powder

¹H NMR (600 MHz, DMSO): δ 7.29 (s, 1H), 7.19 (t, J=7.7 Hz, 1H), 7.10 (d, J=7.6 Hz, 2H), 5.98 (s, 1H), 4.34 (s, 1H), 3.40 (d, J=5.5 Hz, 2H), 3.14 (hept, J=7.0 Hz, 2H), 3.05 (q, J=6.2 Hz, 2H), 1.43 (s, 4H), 1.12 (d, J=6.9 Hz, 12H).

¹³C NMR (151 MHz, DMSO): δ 157.1, 146.8, 133.3, 126.8, 122.7, 60.5, 39.7, 29.8, 27.8, 26.7, 23.3.

### Example 13: Compound 11

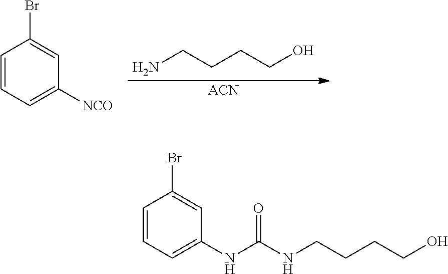

General procedure 1: 3-Bromophenyl isocyanate (125 μL, 1.00 mmol, 1 equiv.), DCM (10 mL, 0.1 M) and 4-aminobutanol (92 μL, 1.00 mmol, 1 equiv.). White powder.

¹H NMR (600 MHz, DMSO): δ 8.06 (d, J=8.3 Hz, 1H), 7.78 (s, 1H), 7.54 (dt, J=8.0, 1.6 Hz, 1H), 7.29-7.23 (m, 1H), 7.06 (t, J=5.6 Hz, 1H), 6.90-6.84 (m, 1H), 4.42 (t, J=5.2 Hz, 1H), 3.45-3.39 (m, 2H), 3.09 (tt, J=6.5, 3.3 Hz, 2H), 1.46 (tt, J=4.9, 1.9 Hz, 4H).

¹³C NMR (151 MHz, DMSO): δ 154.8, 137.9, 132.3, 127.9, 123.0, 121.4, 112.0, 60.5, 39.0, 29.9, 26.2.

### Example 14: Compound 12

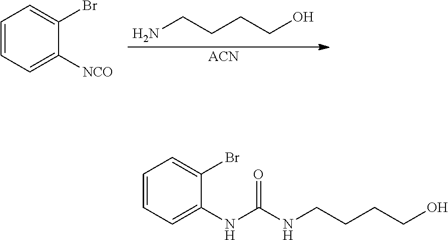

General procedure 1: 2-Bromophenyl isocyanate (130 μL, 1.00 mmol, 1 equiv.), ACN (10 mL, 0.1 M) and 4-aminobutanol (92 μL, 1.00 mmol, 1 equiv.). White powder.

¹H NMR (600 MHz, DMSO): δ 8.63 (s, 1H), 7.81 (t, J=2.0 Hz, 1H), 7.21 (ddd, J=8.2, 2.1, 1.1 Hz, 1H), 7.15 (t, J=8.0 Hz, 1H), 7.04 (ddd, J=7.8, 2.0, 1.0 Hz, 1H), 6.21 (t, J=5.7 Hz, 1H), 4.41 (s, 1H), 3.44-3.38 (m, 2H), 3.08 (q, J=6.4 Hz, 2H), 1.44 (qt, J=4.0, 2.1 Hz, 4H).

¹³C NMR (151 MHz, DMSO): δ 155.0, 142.3, 130.5, 123.4, 121.7, 119.8, 116.3, 60.5, 39.0, 29.9, 26.4.

### Example 15: Compound 13

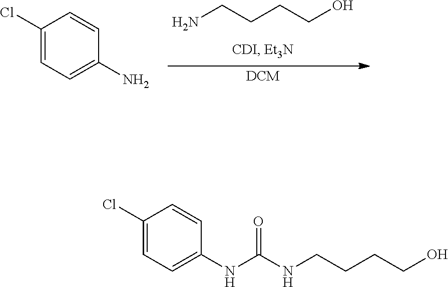

4-Choroaniline (250 mg, 1.96 mmol, 1 equiv.) was dissolved in DCM (10 mL, 0.2 M) and CDI (350 mg, 2.16 mmol, 1.1 equiv.) was added in one portion and stirred for 30 mins. The solution was cooled to 0° C. and Et₃N (1.6 mL, 11.76 mmol, 6 equiv.) was added dropwise followed by 4-aminobutanol (198 μL, 2.16 mmol, 1.1 equiv.). The solution was stirred overnight, and the solution was diluted with EtOAc (25 mL). The mixture was washed with sat. NaHCO₃ (25 mL), 1 M HCl (25 mL) brine (25 mL), dried over MgSO₄, filtered and the solvent was removed under reduced pressure. The residue was pure without purification, producing a white powder.

¹H NMR (600 MHz, DMSO): δ 8.53 (s, 1H), 7.46-7.36 (m, 2H), 7.29-7.19 (m, 2H), 6.14 (t, J=5.7 Hz, 1H), 4.41 (t, J=5.1 Hz, 1H), 3.40 (q, J=5.6 Hz, 2H), 3.07 (d, J=6.0 Hz, 2H), 1.43 (hept, J=4.3 Hz, 4H).

¹³C NMR (151 MHz, DMSO): δ 155.0, 139.6, 128.4, 124.3, 119.0, 60.5, 39.0, 29.9, 26.4.

### Example 16: Compound 14

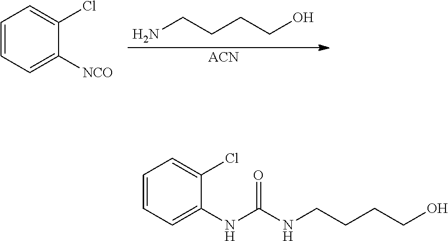

General procedure 1: 2-Chlorophenyl isocyanate (121 μL, 1.00 mmol, 1 equiv.), ACN (10 mL, 0.1 M) and 4-aminobutanol (92 μL, 1.00 mmol, 1 equiv.). White powder.

¹H NMR (600 MHz, DMSO): δ 8.16 (dd, J=8.4, 1.6 Hz, 1H), 7.97 (s, 1H), 7.37 (dd, J=8.0, 1.6 Hz, 1H), 7.22 (ddd, J=8.6, 7.4, 1.6 Hz, 1H), 7.00 (t, J=5.6 Hz, 1H), 6.92 (td, J=7.6, 1.6 Hz, 1H), 4.43 (t, J=5.1 Hz, 1H), 3.42 (d, J=5.3 Hz, 2H), 3.10 (td, J=5.8, 2.4 Hz, 2H), 1.46 (p, J=3.2 Hz, 4H).

¹³C NMR (151 MHz, DMSO): δ 154.8, 136.9, 129.0, 127.4, 122.3, 121.0, 120.7, 60.5, 39.0, 30.0, 26.3.

### Example 17: Compound 15

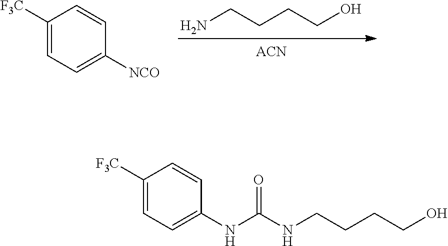

General procedure 1: 4-Trifluoromethanephenyl isocyanate (100 μL, 0.70 mmol, 1 equiv.), ACN (10 mL, 0.1 M) and 4-aminobutanol (65 μL, 0.70 mmol, 1 equiv.). White powder.

¹H NMR (600 MHz, DMSO): δ 8.83 (s, 1H), 7.60-7.52 (m, 5H), 6.27 (t, J=5.7 Hz, 1H), 4.42 (t, J=5.1 Hz, 1H), 3.44-3.38 (m, 2H), 3.09 (q, J=6.4 Hz, 2H), 1.50-1.39 (m, 4H).

¹³C NMR (151 MHz, DMSO): δ 154.8, 144.3, 127.4, 125.95 (q, J=3.7 Hz), 125.6, 123.8, 122.0, 121.2, 121.0, 120.85 (q, J=31.9 Hz), 117.1, 60.5, 39.0, 29.9, 26.4.

### Example 18: Compound 16

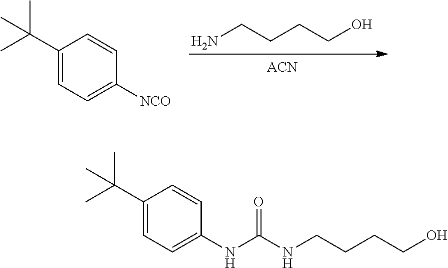

General procedure 1: 4-tertButylphenyl isocyanate (100 μL, 0.563 mmol, 1 equiv.), ACN (5.6 mL, 0.1 M) and 4-aminobutanol (52 μL, 0.563 mmol, 1 equiv.). White powder.

¹H NMR (600 MHz, DMSO): δ 8.27 (s, 1H), 7.30-7.25 (m, 2H), 7.24-7.19 (m, 2H), 6.03 (t, J=5.7 Hz, 1H), 4.41 (t, J=5.1 Hz, 1H), 3.44-3.37 (m, 2H), 3.09-3.03 (m, 2H), 1.47-1.39 (m, 4H), 1.23 (s, 9H).

¹³C NMR (151 MHz, DMSO): δ 155.3, 143.1, 138.0, 125.2, 117.4, 60.5, 39.0, 33.8, 31.3, 29.9, 26.5.

### Example 19: Compound 17

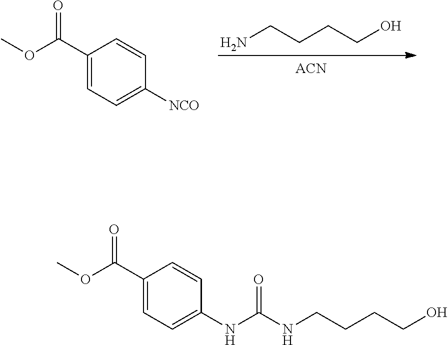

General procedure 1: Methyl-4-phenylisocyanate (100 mg, 0.564 mmol, 1 equiv.), ACN (5.6 mL, 0.1 M) and 4-aminobutanol (52 μL, 0.563 mmol, 1 equiv.). White powder.

¹H NMR (600 MHz, DMSO): δ 8.84 (s, 1H), 7.85-7.79 (m, 2H), 7.53-7.48 (m, 2H), 6.28 (t, J=5.7 Hz, 1H), 4.41 (t, J=5.1 Hz, 1H), 3.79 (s, 3H), 3.41 (td, J=6.2, 5.1 Hz, 2H), 3.12-3.06 (m, 2H), 1.55-1.34 (m, 3H).

¹³C NMR (151 MHz, DMSO): δ 166.0, 154.7, 145.3, 130.4, 121.5, 116.6, 60.5, 51.7, 39.0, 29.9, 26.3.

### Example 20: Compound 18

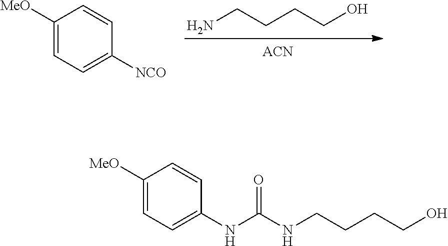

General procedure 1: 4-Methoxyphenyl isocyanate (130 μL, 1.00 mmol, 1 equiv.), ACN (10 mL, 0.1 M) and 4-aminobutanol (92 μL, 1.00 mmol, 1 equiv.). White powder

¹H NMR (600 MHz, DMSO): δ 8.16 (s, 1H), 7.29-7.24 (m, 2H), 6.82-6.77 (m, 2H), 5.98 (t, J=5.7 Hz, 1H), 4.40 (t, J=5.1 Hz, 1H), 3.68 (s, 3H), 3.39 (t, J=5.6 Hz, 2H), 3.05 (q, J=6.0 Hz, 2H), 1.43 (p, J=3.3 Hz, 4H).

¹³C NMR (151 MHz, DMSO): δ 155.4, 153.8, 133.8, 119.3, 113.9, 60.5, 55.1, 39.0, 29.9, 26.5.

### Example 21: Compound 19

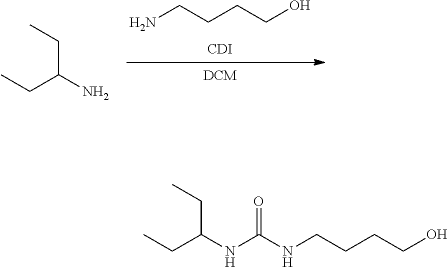

General procedure 3: 3-Aminopentante (118 μL, 1.01 mmol, 1 equiv.), CDI (197 mg, 1.21 mmol, 1.2 equiv.), THF (5 mL, 0.2 M) and 4-aminobutanol (102 μL, 1.11 mmol, 1.1 equiv.). Recrystallization, white crystals.

: 3-Aminopentante (118 μL, 1.01 mmol, 1 equiv.) was dissolved in DCM (5 mL, 0.2 M) and CDI (197 mg, 1.21 mmol, 1.2 equiv.) was added in one portion and stirred for 30 mins. The solution was cooled to 0° C. and 4-aminobutanol (102 μL, 1.11 mmol, 1.1 equiv.). The solution was stirred overnight, and the solution was diluted with EtOAc (25 mL). The mixture was washed with sat. NaHCO₃ (25 mL), 1 M HCl (25 mL) brine (25 mL), dried over MgSO₄, filtered and the solvent was removed under reduced pressure. The residue was recrystallized in Hexanes overnight to afford clear crystals.

¹H NMR (600 MHz, DMSO): δ 5.64 (s, 1H), 5.49 (d, J=8.7 Hz, 1H), 4.37 (t, J=5.1 Hz, 1H), 3.38 (td, J=6.1, 5.0 Hz, 2H), 2.96 (td, J=6.6, 5.5 Hz, 2H), 1.43-1.32 (m, 6H), 1.25 (dp, J=13.4, 7.5 Hz, 2H), 0.80 (t, J=7.4 Hz, 6H).

¹³C NMR (151 MHz, DMSO): δ 158.1, 60.5, 51.4, 39.6, 29.9, 27.3, 26.8, 10.2.

### Example 22: Compound 20

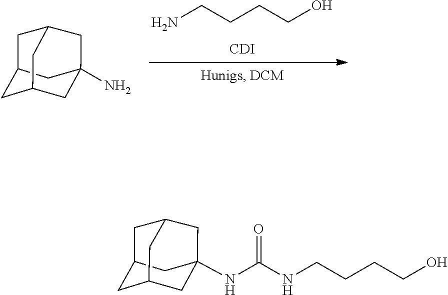 [SMILES: NCCCCO ; NC12CC3CC(CC(C3)C1)C2 ; O=C(NCCCCO)NC12CC3CC(CC(C3)C1)C2]

General procedure 3: Adamantyl amine (65 mg, 0.432 mmol, 1 equiv.) was dissolved in DCM (2.1 mL, 0.2 M) and CDI (77 mg, 0.475 mmol, 1.1 equiv.) was added in one portion and stirred for 30 mins. The solution was cooled to 0° C. and DIEA (450 μL, 2.59 mmol, 6 equiv.) was added dropwise followed by 4-aminobutanol (44 μL, 0.475 mmol, 1.1 equiv.). The solution was stirred overnight, and the solution was diluted with EtOAc (25 mL). The mixture was washed with sat. NaHCO₃ (25 mL), 1 M HCl (25 mL) brine (25 mL), dried over MgSO₄, filtered and the solvent was removed under reduced pressure. The residue was pure without purification, producing an off-white powder.

¹H NMR (600 MHz, DMSO): δ 5.59 (t, J=5.7 Hz, 1H), 5.43 (s, 1H), 4.37 (t, J=5.1 Hz, 1H), 3.37 (td, J=6.2, 4.9 Hz, 2H), 2.90 (td, J=6.7, 5.5 Hz, 2H), 1.98 (d, J=3.0 Hz, 2H), 1.84 (d, J=2.9 Hz, 6H), 1.59 (q, J=3.4 Hz, 7H), 1.42-1.28 (m, 4H).

¹³C NMR (151 MHz, DMSO): δ 157.1, 60.5, 49.3, 42.1, 38.7, 36.1, 30.0, 28.9, 26.7.

### Example 23: Compound 21

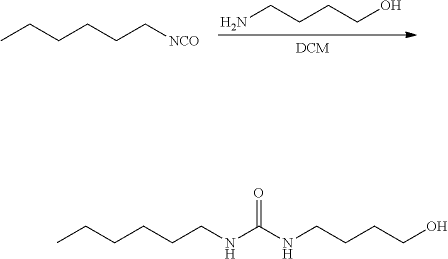

General procedure 1: Hexyl isocyanate (50 μL, 0.45 mmol, 1 equiv.), DCM (2.2 mL, 0.2 M) and 4-aminobutanol (42 μL, 0.45 mmol, 1 equiv.). White powder

¹H NMR (400 MHz, DMSO): δ 5.74 (t, J=5.7 Hz, 2H), 3.47-3.33 (m, 10H), 2.99-2.90 (m, 4H), 1.44-1.14 (m, 7H), 0.85 (t, J=6.7 Hz, 3H).

¹³C NMR (151 MHz, DMSO): δ 158.2, 60.6, 39.3, 39.2, 31.1, 30.1, 29.9, 26.8, 26.1, 22.1, 13.9.

### Example 24: Compound 22

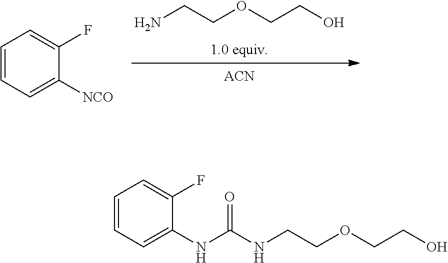

General procedure 2: 2-Fluorophenyl isocyanate (163 μL, 1.46 mmol, 1 equiv.), ACN (29 mL, 0.05 M) and 2(2-aminoethoxy)ethanol (161 μL, 1.6 mmol, 1 equiv.). Flash column chromatography (50% EtOAc in Hexanes), white powder.

¹H NMR (400 MHz, CDCl₃): δ 8.01 (td, J=8.2, 1.7 Hz, 1H), 7.72 (d, J=2.8 Hz, 1H), 7.04-6.83 (m, 3H), 6.46 (t, J=5.5 Hz, 1H), 4.06 (m, 1H), 3.69 (q, J=4.7 Hz, 2H), 3.52 (ddd, J=6.8, 3.6, 1.8 Hz, 4H), 3.40 (q, J=5.2 Hz, 2H).

¹³C NMR (101 MHz, CDCl₃): δ 156.4, 152.7 (d, J=242.0 Hz), 127.6 (d, J=10.3 Hz), 124.4 (d, J=3.6 Hz), 122.7 (d, J=7.4 Hz), 121.5, 114.8 (d, J=19.3 Hz), 72.2, 70.3, 61.5, 39.8.

### Example 25: Compound 23

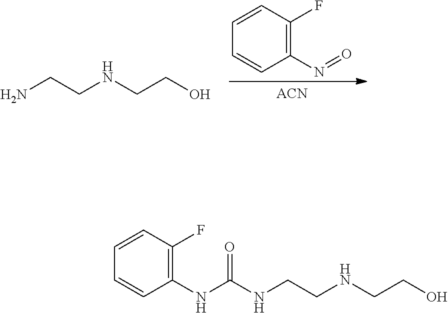 [SMILES: O=Nc1ccccc1F ; NCCNCCO ; O=C(NCCNCCO)Nc1ccccc1F]

General procedure 2: 2-Fluorophenyl isocyanate (143 μL, 0.867 mmol, 1 equiv.), ACN (17 mL, 0.05 M) and 2(2-aminoethoxy)ethanol (88 μL, 0.867 mmol, 1 equiv.). Flash column chromatography (50-100% EtOAc in Hexanes), white powder.

¹H NMR (600 MHz, DMSO): δ 8.37 (s, 1H), 8.11 (td, J=8.3, 1.7 Hz, 1H), 7.15 (ddd, J=11.8, 8.2, 1.4 Hz, 1H), 7.06 (t, J=8.0 Hz, 1H), 6.90 (tdd, J=7.9, 5.0, 1.6 Hz, 1H), 6.70 (t, J=5.6 Hz, 1H), 4.48 (s, 1H), 3.46 (t, J=5.7 Hz, 2H), 3.16 (q, J=5.9 Hz, 2H), 2.61 (dt, J=9.3, 5.9 Hz, 4H).

¹³C NMR (151 MHz, DMSO): δ 155.0, 151.6 (d, J=240.8 Hz), 128.4 (d, J=10.3 Hz), 124.3 (d, J=3.3 Hz), 121.4 (d, J=7.3 Hz), 120.2, 114.8 (d, J=19.0 Hz), 60.3, 51.4, 49.0, 39.6.

### Example 26: Compound 24

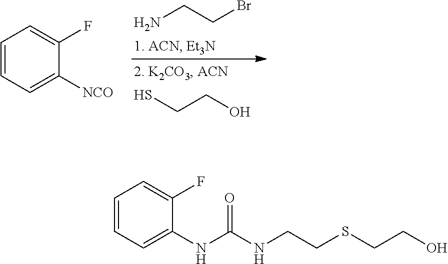

Ethyl bromide 2-fluorophenyl urea (150 mg, 0.575 mmol, 1 equiv.) was dissolved in ACN (5.7 mL, 0.1 M) and K₂CO₃ (80 mg, 0.575 mmol, 1 equiv.) was added followed by β-mercaptoethanol (40 μL, 0.575 mmol, 1 equiv.). The mixture was stirred at room temperature overnight. The mixture was filtered, and the volatiles were removed under reduced pressure. The crude residue was purified by flash column chromatography (25-50% EtOAc in Hexanes) to provide the product as a clear oil.

¹H NMR (600 MHz, CDCl₃): δ 7.96 (td, J=8.2, 1.7 Hz, 1H), 7.52 (d, J=2.8 Hz, 1H), 7.05-6.97 (m, 2H), 6.95-6.86 (m, 1H), 6.31 (t, J=5.9 Hz, 1H), 4.40 (t, J=8.1 Hz, 1H), 3.69 (t, J=6.0 Hz, 2H), 3.37 (q, J=6.3 Hz, 2H), 2.67 (t, J=6.0 Hz, 2H), 2.61 (t, J=6.5 Hz, 2H).

¹³C NMR (151 MHz, CDCl₃): δ 156.0, 153.0 (d, J=242.5 Hz), 127.4 (d, J=10.4 Hz), 124.5 (d, J=3.5 Hz), 123.1 (d, J=7.5 Hz), 121.9, 115.0 (d, J=19.2 Hz), 61.0, 39.7, 35.2, 32.6.

### Example 27: Compound 25

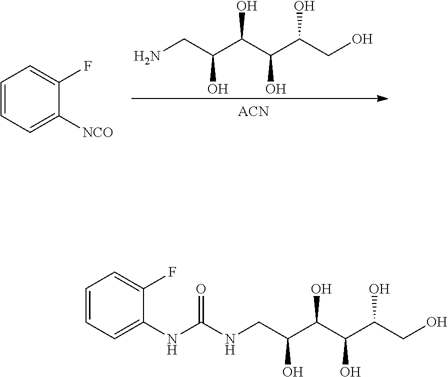

General procedure 1: 2-Fluorophenyl isocyanate (81.5 μL, 0.73 mmol, 1 equiv.), ACN (14 mL, 0.05 M) and D-glucamine (145 mg, 0.8 mmol, 1.1 equiv.). Off-white powder.

¹H NMR (400 MHz, D₂O): δ 7.57-7.46 (m, 1H), 7.22-7.10 (m, 3H), 3.87 (dt, J=7.6, 4.8 Hz, 1H), 3.82-3.71 (m, 3H), 3.70-3.58 (m, 2H), 3.44 (dd, J=14.2, 4.3 Hz, 1H), 3.31 (d, J=0.8 Hz, 1H), 3.25 (dd, J=14.2, 7.5 Hz, 1H).

¹³C NMR (101 MHz, DMSO): δ 154.9, 151.6 (d, J=240.5 Hz), 128.5 (d, J=10.2 Hz), 124.3 (d, J=3.3 Hz), 121.3 (d, J=7.4 Hz), 120.1, 114.7 (d, J=19.0 Hz), 51.8, 46.3, 38.9, 27.6, 23.8, 11.4.

### Example 28: Compound 26

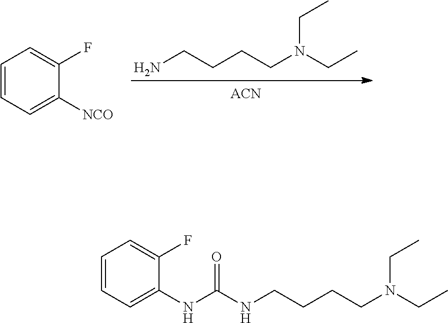

General procedure 2: 2-Fluorophenyl isocyanate (100 μL, 0.81 mmol, 1 equiv.), ACN (16.2 mL, 0.05 M) and N,N′-Diethylamine (128 mg, 0.891 mmol, 1.1 equiv.). Flash column chromatography (50-100% EtOAc in Hexanes), clear yellow oil.

¹H NMR (600 MHz, DMSO): δ 8.32 (d, J=2.8 Hz, 1H), 8.19 (td, J=8.3, 1.7 Hz, 1H), 7.18 (ddd, J=11.7, 8.2, 1.5 Hz, 1H), 7.09 (ddd, J=8.1, 7.3, 1.3 Hz, 1H), 6.93 (dddd, J=8.2, 7.5, 5.1, 1.7 Hz, 1H), 6.75 (t, J=5.6 Hz, 1H), 3.19-3.09 (m, 1H), 2.52 (q, J=7.1 Hz, 4H), 2.47-2.43 (m, 2H), 1.46 (p, J=3.5 Hz, 4H), 0.99 (t, J=7.2 Hz, 6H).

¹³C NMR (101 MHz, DMSO): δ 155.3, 152.0 (d, J=240.5 Hz), 128.9 (d, J=10.2 Hz), 124.8 (d, J=3.3 Hz), 121.8 (d, J=7.4 Hz), 120.5, 115.2 (d, J=19.0 Hz), 52.3, 46.7, 28.0, 24.2, 11.9.

### Example 29: Compound 27

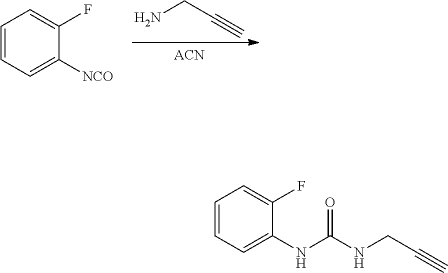

General procedure 1: 2-Fluorophenyl isocyanate (150 μL, 1.33 mmol, 1 equiv.), ACN (13.3 mL, 0.1 M) and propargylamine (85 μL, 1.33 mmol, 1 equiv.). White powder.

¹H NMR (600 MHz, DMSO): δ 8.39 (d, J=2.7 Hz, 1H), 8.06 (td, J=8.3, 1.7 Hz, 1H), 7.17 (ddd, J=11.7, 8.2, 1.5 Hz, 1H), 7.08 (td, J=7.8, 1.5 Hz, 1H), 6.97-6.92 (m, 1H), 6.90 (t, J=5.7 Hz, 1H), 3.90 (dd, J=5.6, 2.5 Hz, 2H), 3.11 (td, J=2.5, 0.6 Hz, 1H).

¹³C NMR (151 MHz, DMSO): δ 154.6, 151.8 (d, J=241.1 Hz), 127.9 (d, J=10.3 Hz), 124.5 (d, J=3.3 Hz), 122.2 (d, J=7.4 Hz), 120.6 (d, J=1.9 Hz), 114.9 (d, J=19.0 Hz), 81.8, 73.1, 28.8.

### Example 30: Compound 28

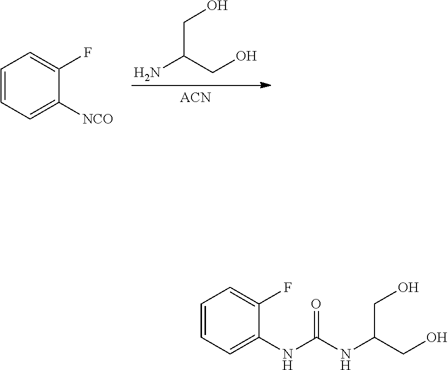

General procedure 1: 2-Fluorophenyl isocyanate (150 μL, 1.33 mmol, 1 equiv.), ACN (13.3 mL, 0.1 M) and serinol (121 mg, 1.33 mmol, 1 equiv.). White powder.

¹H NMR (600 MHz, DMSO): δ 8.44 (d, J=2.7 Hz, 1H), 8.13 (td, J=8.3, 1.7 Hz, 1H), 7.15 (ddd, J=11.7, 8.2, 1.5 Hz, 1H), 7.06 (td, J=7.8, 1.4 Hz, 1H), 6.90 (dddd, J=8.1, 7.4, 5.1, 1.7 Hz, 1H), 6.67 (d, J=8.1 Hz, 1H), 4.71 (s, 2H), 3.61 (dddd, J=8.0, 6.0, 4.5, 1.4 Hz, 1H), 3.48 (dd, J=10.5, 4.6 Hz, 2H), 3.41 (dd, J=10.5, 6.0 Hz, 2H).

¹³C NMR (151 MHz, DMSO): δ 154.7, 151.5 (d, J=240.6 Hz), 128.4 (d, J=10.3 Hz), 124.3 (d, J=3.3 Hz), 121.3 (d, J=7.4 Hz), 120.1, 114.7 (d, J=19.0 Hz), 60.1, 52.6.

### Example 31: Compound 29

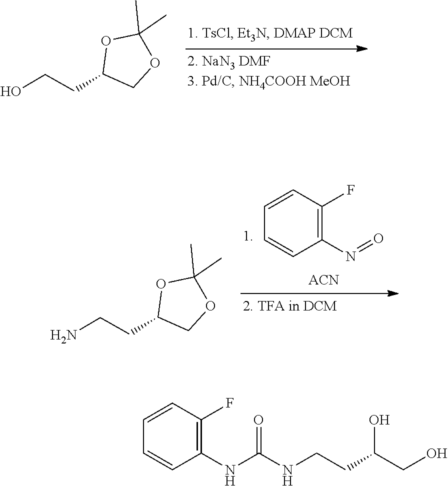

### Deprotection Compound 29

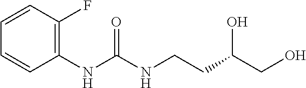 [SMILES: O=C(NCC[C@H](O)CO)Nc1ccccc1F]

Protected urea (51 mg, mmol, 1 equiv) was dissolved in DCM (0.5 M) and TFA (500 μL) was added. The solution was stirred at room temperature for 1 h and the volatiles were removed under reduced pressure. The residue was purified by flash column chromatography (5-10% MeOH in DCM), affording pure urea product as a white powder (27 mg, yield)

¹H NMR (600 MHz, DMSO): δ 8.28 (d, J=2.7 Hz, 1H), 8.11 (td, J=8.3, 1.7 Hz, 1H), 7.15 (ddd, J=11.8, 8.2, 1.5 Hz, 1H), 7.09-7.02 (m, 1H), 6.90 (dddd, J=8.2, 7.4, 5.1, 1.7 Hz, 1H), 6.59 (t, J=5.6 Hz, 1H), 4.56-4.47 (m, 2H), 3.47 (dtd, J=9.1, 5.5, 3.6 Hz, 1H), 3.31 (d, J=5.5 Hz, 1H), 3.27-3.19 (m, 2H), 3.14 (dtd, J=13.1, 7.4, 5.5 Hz, 1H), 1.62 (dtd, J=13.5, 7.7, 3.6 Hz, 1H), 1.35 (dddd, J=13.5, 9.0, 7.2, 5.4 Hz, 1H).

¹³C NMR (151 MHz, DMSO): δ 154.9, 151.6 (d, J=240.3 Hz), 128.4 (d, J=10.2 Hz), 124.3 (d, J=3.3 Hz), 121.3 (d, J=7.5 Hz), 120.1, 114.7 (d, J=19.0 Hz), 69.1, 65.9, 36.1, 33.8.

### Example 32: Compound 30

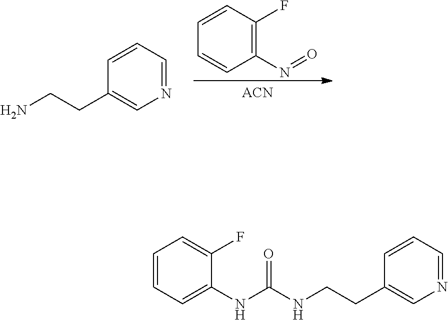

General procedure 2: 2-Fluorophenyl isocyanate (112 μL, 1.00 mmol, 1 equiv.), ACN (10 mL, 0.1 M) and 3-(2-Aminoethyl)pyridine (117 μL, 1.00 mmol, 1 equiv.). Flash column chromatography (100% EtOAc), clear oil.

¹H NMR (600 MHz, CDCl₃): δ 8.30-8.24 (m, 2H), 8.02 (td, J=8.2, 1.7 Hz, 1H), 7.95 (d, J=2.7 Hz, 1H), 7.46 (dt, J=7.9, 2.0 Hz, 1H), 7.11 (ddd, J=7.8, 4.8, 0.9 Hz, 1H), 7.00 (td, J=7.8, 1.6 Hz, 1H), 6.92 (ddd, J=11.2, 8.2, 1.6 Hz, 1H), 6.86 (dddd, J=8.2, 7.0, 5.1, 1.7 Hz, 1H), 6.55 (t, J=5.8 Hz, 1H), 3.44 (q, J=6.6 Hz, 2H), 2.73 (t, J=6.9 Hz, 2H).

¹³C NMR (151 MHz, CDCl₃): δ 156, 152.7 (d, J=242.3 Hz), 149.7, 147.4, 136.7, 135.0, 127.6 (d, J=10.4 Hz), 124.4 (d, J=3.5 Hz), 123.7, 122.7 (d, J=7.3 Hz), 121.5 (d, J=1.5 Hz), 114.8 (d, J=19.2 Hz), 40.7, 33.3.

### Example 33: Compound 31

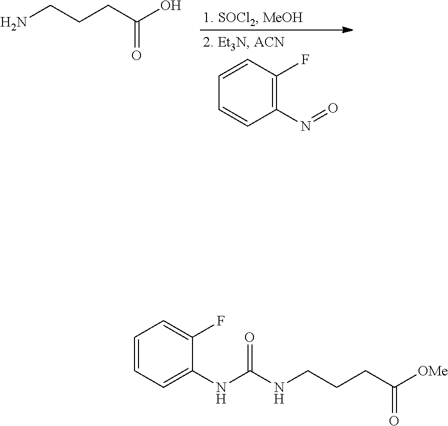 [SMILES: NCCCC(=O)O ; O=Nc1ccccc1F ; COC(=O)CCCNC(=O)Nc1ccccc1F]

4-Amino butyric acid (192 mg, 1.86 mmol, 1 equiv.) was dissolved in MeOH (2.9 mL, 0.65 M) and the stirring solution was cooled to 0° C. SOCl₂ (0.408 mL, 5.59 mmol, 3 equiv.) was added dropwise (Caution: Gas evolution) and the resulting mixture was stirred overnight at room temperature. The volatiles were removed under reduced pressure and the residue was used directly without further purification.

Crude ester (1.86 mmol, 1 equiv.) was dissolved in ACN (18.6 mL, 0.1 M). Et₃N (0.253 mL, 1.86 mmol, 1 equiv.) was added dropwise followed by 2-flurophenyl isocyanate (0.209 mL, 1.86 mmol, 1 equiv.) and the solution was stirred at room temperature for 2 h. The volatiles were removed under reduced pressure and the residue was purified by flash column chromatography (50% EtOAc in Hexanes) to afford purified methyl ester urea.

¹H NMR (400 MHz, DMSO): δ 8.24 (d, J=2.7 Hz, 1H), 8.10 (td, J=8.3, 1.7 Hz, 1H), 7.16 (ddd, J=11.8, 8.2, 1.5 Hz, 1H), 7.11-7.02 (m, 1H), 6.91 (dddd, J=8.1, 7.2, 5.1, 1.7 Hz, 1H), 6.65 (t, J=5.7 Hz, 1H), 3.59 (s, 3H), 3.10 (td, J=6.8, 5.6 Hz, 2H), 2.34 (t, J=7.4 Hz, 2H), 1.68 (p, J=7.1 Hz, 2H).

¹³C NMR (101 MHz, DMSO): δ 173.6, 155.3, 153.2, 128.77 (d, J=10.0 Hz), 124.82 (d, J=3.4 Hz), 121.94 (d, J=7.3 Hz), 120.6, 115.22 (d, J=19.1 Hz), 51.8, 38.8, 31.2, 25.6.

### Example 34: Compound 32

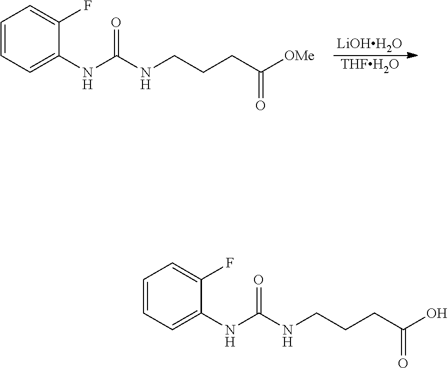

Methyl ester urea (112 mg, 0.440 mmol, 1 equiv.) was dissolved in THF:H₂O (2.2 mL, 0.2 M, 1:1). LiOH·H₂O (37 mg, 0.880 mmol, 2 equiv.) was added and the mixture was stirred at room temperature for 2 h. The volatiles were removed under reduced pressure and the residue was taken up in 1 M HCl solution (25 mL) and was extracted with EtOAc (3×25 mL) and the organic fraction was washed with H₂O (25 mL), dried over MgSO₄, filtered and the solvent removed under reduced pressure, affording purified product as a white solid.

¹H NMR (600 MHz, DMSO): δ 12.09 (s, 1H), 8.23 (d, J=2.7 Hz, 1H), 8.11 (td, J=8.3, 1.7 Hz, 1H), 7.15 (ddd, J=11.8, 8.2, 1.5 Hz, 1H), 7.06 (td, J=7.8, 1.4 Hz, 1H), 6.90 (dddd, J=7.9, 6.8, 5.1, 1.7 Hz, 1H), 6.65 (t, J=5.7 Hz, 1H), 3.11 (q, J=6.8 Hz, 2H), 2.25 (t, J=7.4 Hz, 2H), 1.66 (p, J=7.1 Hz, 2H).

¹³C NMR (151 MHz, DMSO): δ 174.3, 154.9, 151.63 (d, J=240.3 Hz), 128.39 (d, J=10.1 Hz), 124.38 (d, J=3.3 Hz), 121.48 (d, J=7.3 Hz), 120.2, 114.78 (d, J=19.0 Hz), 38.4, 31.1, 25.2.

### Example 35: Compound 33

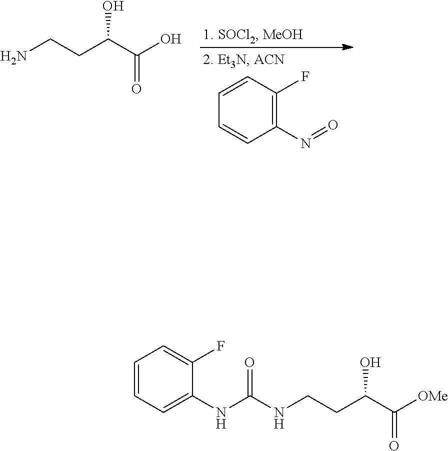 [SMILES: NCC[C@H](O)C(=O)O ; O=Nc1ccccc1F ; COC(=O)[C@@H](O)CCNC(=O)Nc1ccccc1F]

(S)-4-amino-2-hydroxybutanoic acid (244 mg, 2.05 mmol, 1 equiv.) was dissolved in MeOH (3.15 mL, 0.65 M) and the stirring solution was cooled to 0° C. SOCl₂ (449 μL, 6.14 mmol, 3 equiv.) was added dropwise (Caution: Gas evolution) and the resulting mixture was stirred overnight at room temperature. The volatiles were removed under reduced pressure and the residue was used directly without further purification.

Crude ester (2.05 mmol, 1 equiv.) was dissolved in ACN (20.5 mL, 0.1 M). Et₃N (279 μL, 2.05 mmol, 1 equiv.) was added dropwise followed by 2-flurophenyl isocyanate (253 μL, 2.05 mmol, 1 equiv.) and the solution was stirred at room temperature for 2 h. The volatiles were removed under reduced pressure and the residue was purified by flash column chromatography (50-75% EtOAc in Hexanes) to afford purified methyl ester urea.

¹H NMR (400 MHz, DMSO): δ 8.30 (d, J=2.7 Hz, 1H), 8.11 (td, J=8.3, 1.7 Hz, 1H), 7.16 (ddd, J=11.8, 8.2, 1.5 Hz, 1H), 7.11-7.02 (m, 1H), 6.96-6.86 (m, 1H), 6.66 (t, J=5.7 Hz, 1H), 5.53 (d, J=5.9 Hz, 1H), 4.10 (ddd, J=8.7, 5.9, 4.2 Hz, 1H), 3.63 (s, 3H), 3.18 (qd, J=6.7, 3.7 Hz, 2H), 1.82 (dtd, J=14.3, 7.3, 4.1 Hz, 1H), 1.74-1.61 (m, 1H).

¹³C NMR (101 MHz, DMSO): δ 174.8, 155.2, 151.84 (d, J=240.6 Hz), 128.55 (d, J=10.3 Hz), 124.60 (d, J=3.3 Hz), 121.73 (d, J=7.3 Hz), 120.4, 115.01 (d, J=19.1 Hz), 68.0, 51.7, 35.8, 34.4.

### Example 36: Compound 34

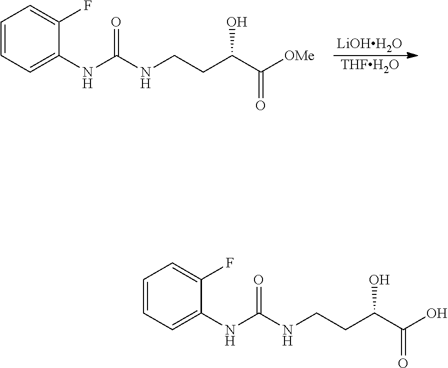

Methyl ester urea (130 mg, 0.481 mmol, 1 equiv.) was dissolved in THF:H₂O (2.4 mL, 0.2M, 1:1). LiOH·H₂O (40.4 mg, 0.962 mmol, 2 equiv.) was added and the mixture was stirred at room temperature for 2 h. The volatiles were removed under reduced pressure and the residue was taken up in 1 M HCl solution (25 mL) and was extracted with EtOAc (3×25 mL) and the organic fraction was washed with H₂O (25 mL), dried over MgSO₄, filtered and the solvent removed under reduced pressure, affording purified product as a white solid.

¹H NMR (600 MHz, DMSO): δ 8.29 (d, J=2.7 Hz, 1H), 8.11 (td, J=8.4, 1.7 Hz, 1H), 7.15 (ddd, J=11.8, 8.1, 1.4 Hz, 1H), 7.09-7.03 (m, 1H), 6.94-6.87 (m, 1H), 6.66 (t, J=5.8 Hz, 1H), 4.00 (dd, J=8.9, 4.0 Hz, 1H), 3.19 (td, J=12.5, 5.9 Hz, 2H), 1.91 (q, J=0.9 Hz, 1H), 1.83 (dtd, J=13.4, 7.5, 4.0 Hz, 1H), 1.69-1.60 (m, 1H).

¹³C NMR (151 MHz, DMSO): δ 175.8, 155.0, 151.64 (d, J=240.5 Hz), 128.39 (d, J=10.2 Hz), 124.39 (d, J=3.3 Hz), 121.50 (d, J=7.2 Hz), 120.2, 114.80 (d, J=18.9 Hz), 67.7, 35.8, 34.3.

### Example 37: Compound 35

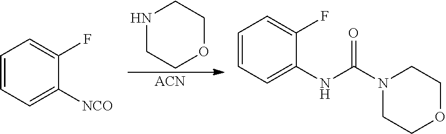

General procedure 1: 2-Fluorophenyl isocyanate (150 μL, 1.33 mmol, 1 equiv.), ACN (13.3 mL, 0.1 M) and morpholine (115 μL, 1.33 mmol, 1 equiv.). Off-white powder.

¹H NMR (600 MHz, DMSO): δ 8.31 (s, 1H), 7.46-7.39 (m, 1H), 7.21-7.14 (m, 1H), 7.14-7.08 (m, 2H), 3.62-3.57 (m, 4H), 3.40 (d, J=4.6 Hz, 4H).

¹³C NMR (151 MHz, DMSO): δ 156.2, 155.4 (d, J=244.9 Hz), 127.5 (d, J=11.6 Hz), 126.3 (d, J=1.9 Hz), 125.2 (d, J=7.6 Hz), 124.1 (d, J=3.4 Hz), 115.5 (d, J=20.0 Hz), 66.0, 44.3.

### Example 38: Compound 36

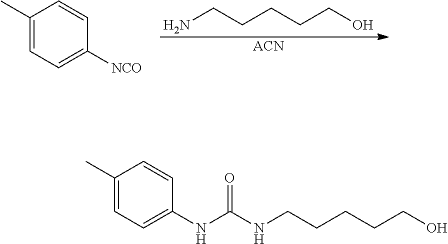

General procedure 1: 4-Methylphenyl isocyanate (143 μL, 1.14 mmol, 1 equiv.), ACN (11 mL, 0.1 M) and 5-aminobutanol (156 mg, 1.14 mmol, 1 equiv.). Off-white powder.

¹H NMR (600 MHz, DMSO): δ 8.24 (s, 1H), 7.25 (d, J=8.4 Hz, 2H), 7.03-6.96 (m, 2H), 6.03 (t, J=5.7 Hz, 1H), 4.38-4.32 (m, 1H), 3.39 (td, J=6.5, 5.1 Hz, 2H), 3.05 (td, J=6.9, 5.7 Hz, 2H), 2.20 (s, 3H), 1.47-1.36 (m, 4H), 1.34-1.26 (m, 2H).

¹³C NMR (151 MHz, DMSO): δ 155.3, 138.0, 129.5, 129.0, 117.6, 60.7, 32.3, 29.7, 23.0, 20.3.

### Example 39: Compound 37

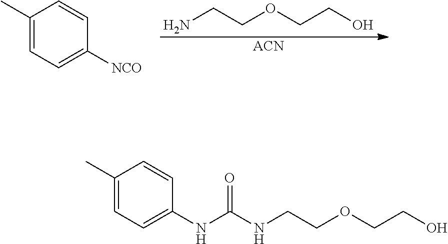

General procedure 2: 4-Methyl phenyl isocyanate (114 μL, 0.906 mmol, 1 equiv.), ACN (18.12 mL, 0.05 M) and 2(2-aminoethoxy)ethanol (100 μL, 0.906 mmol, 1 equiv.). Flash column chromatography (50% EtOAc in Hexanes), white powder.

¹H NMR (600 MHz, CDCl₃): δ 7.69 (s, 1H), 7.15 (d, J=8.4 Hz, 2H), 7.03-6.98 (m, 2H), 6.13 (t, J=5.7 Hz, 1H), 4.07 (s, 1H), 3.69 (d, J=4.6 Hz, 2H), 3.55-3.45 (m, 4H), 3.37 (q, J=5.3 Hz, 2H), 2.25 (s, 3H).

¹³C NMR (151 MHz, CDCl₃): δ 157.1, 136.5, 132.4, 129.5, 120.1, 72.2, 70.4, 61.5, 39.8, 20.8.

### Example 40: Compound 38

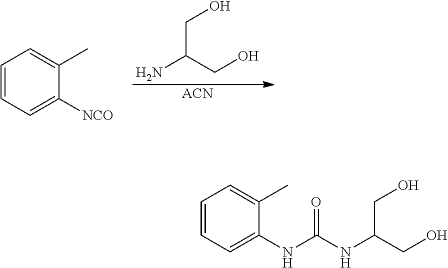

General procedure 1: 2-Methylphenyl isocyanate (150 μL, 1.2 mmol, 1 equiv.), ACN (12 mL, 0.1 M) and serinol (110 mg, 1.2 mmol, 1 equiv.). White powder.

¹H NMR (600 MHz, DMSO): δ 7.86 (dd, J=8.2, 1.3 Hz, 1H), 7.79 (s, 1H), 7.12-7.01 (m, 2H), 6.84 (td, J=7.4, 1.3 Hz, 1H), 6.58 (d, J=8.0 Hz, 1H), 4.71 (t, J=5.3 Hz, 2H), 3.61 (dddd, J=10.5, 8.6, 6.0, 4.5 Hz, 1H), 3.51 (dt, J=10.5, 4.7 Hz, 2H), 3.42 (dt, J=10.4, 5.9 Hz, 2H), 2.17 (s, 3H).

¹³C NMR (151 MHz, DMSO): δ 155.2, 138.4, 130.0, 126.3, 126.0, 121.5, 120.1, 60.2, 52.6, 18.0.

### Example 41: Compound 39

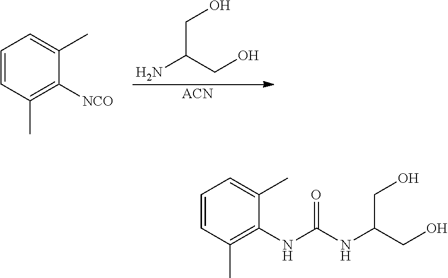

General procedure 1: 2,6-Dimethylphenyl isocyanate (150 μL, 1.08 mmol, 1 equiv.), ACN (10 mL, 0.1 M) and serinol (98 mg, 1.08 mmol, 1 equiv.). White powder.

¹H NMR (600 MHz, DMSO): δ 7.62 (s, 1H), 7.04-6.97 (m, 4H), 5.90 (s, 1H), 4.69 (dd, J=5.7, 4.8 Hz, 2H), 3.56 (dtt, J=8.5, 6.1, 4.5 Hz, 1H), 3.49 (dt, J=10.3, 4.6 Hz, 2H), 3.39 (dt, J=10.3, 5.9 Hz, 2H), 2.14 (s, 7H).

¹³C NMR (151 MHz, DMSO): δ 155.8, 136.1, 135.4, 127.6, 125.5, 60.2, 52.7, 18.2.

### Example 42: Compound 40

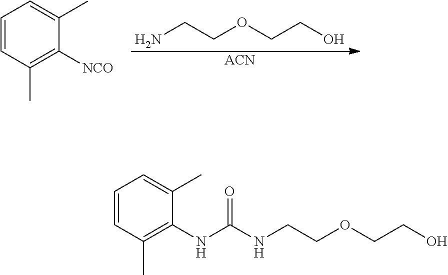

General procedure 1: 2-Methylphenyl isocyanate (65 μL, 0.359 mmol, 1 equiv.), ACN (3.6 mL, 0.1 M) and 2(2-aminoethoxy)ethanol (49 mg, 0.359 mmol, 1 equiv.). White powder.

¹H NMR (600 MHz, DMSO): δ 7.52 (s, 1H), 7.01 (q, J=5.1 Hz, 3H), 6.04 (s, 1H), 4.60-4.56 (m, 1H), 3.51 (q, J=5.4 Hz, 2H), 3.44 (td, J=5.5, 2.0 Hz, 4H), 3.21 (q, J=5.7 Hz, 2H), 2.14 (s, 6H).

¹³C NMR (151 MHz, DMSO): δ 155.9, 136.0, 135.5, 127.6, 125.6, 72.2, 70.0, 60.2, 40.1, 18.2.

### Example 43: Compound 41

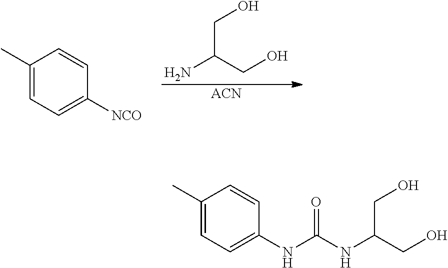

General procedure 1: 4-Methylphenyl isocyanate (150 μL, 1.2 mmol, 1 equiv.), ACN (12 mL, 0.1 M) and serinol (110 mg, 1.2 mmol, 1 equiv.). White powder.

¹H NMR (400 MHz, DMSO): δ 8.49 (s, 1H), 7.27-7.21 (m, 2H), 7.05-6.97 (m, 2H), 5.99 (d, J=8.0 Hz, 1H), 4.73 (t, J=5.2 Hz, 2H), 3.59 (dtt, J=8.1, 5.9, 4.5 Hz, 1H), 3.49 (dt, J=10.5, 4.6 Hz, 2H), 3.44-3.34 (m, 2H), 2.20 (s, 3H).

¹³C NMR (101 MHz, DMSO): δ 155.1, 138.1, 129.6, 129.1, 117.5, 60.2, 52.4, 20.3.

### Example 44: Ice Recrystallization Activity (IC₅₀ Assay)

A splat cooling assay was the initial step in the study to provide the ice recrystallization activity of compounds in the library. The details of the assay used in this study have been previously published (Abraham S.; Keillor, K.; Capicciotti, C. J.; Perley-Robertson, G. E.; Keillor, J. W.; Ben, R. N. Quantitative Analysis of the Efficacy and Potency of Novel Small Molecule Ice Recrystallization Inhibitors. Cryst. Growth Des. 2015, 15, 5034-5039, incorporated herein by reference) and shown to be appropriate for providing accurate ice recrystallization activity data.

Initial compound selection screen was performed using a modified version of the splat cooling assay, where each compound was solubilized at 10 mM in PBS and the Vₙₒᵣₘ was compared to the PBS control defined at 100. Compound IC₅₀'s were fully determined using the following method if the Vₙₒᵣₘ at 10 mM showed complete ice recrystallization inhibition on the lower plateau at 0.

Six solutions were initially prepared with a range of concentrations of the compound of interest, with a base starting range (0.25, 0.5, 1, 5 and 10 mM) solubilized in PBS. Varying IRI activity of compounds may require expanding the concentration range. The upper plateau of the curve was defined at 100 by the control sample, and the lower plateau at 0 by the maximum inhibitor concentration. The IC₅₀ value were then determined by nonlinear regression using a 4-parameter sigmoidal dose-response fitting equation. The lower bound was defined with the assay development in terms of what is considered rate=0; the bottom plateau was not redefined to maximal inhibitor potency. The IC₅₀ value in this case represents the concentration when 50% of the maximal ice recrystallization inhibition was achieved.

### Example 45: Cytotoxicity Analysis (LD₅₀ Determination Method)

A resazurin based assay was used to determine the LD₅₀ and cytotoxicity of the IRI compounds. Resazurin is a cell permeable redox indicator that is used to monitor viable cell number which can be added to cells in culture and viable cells with active metabolism can reduce resazurin to resorufin which is pink and fluorescent, as described previously (Riss T. L.; Moravec R. A.; Niles A. L.; Duellman S.; Benink H. A.; Worzella T. J.; Minor L. Cell Viability Assays. Assay Guidance Manual. Eli Lilly & Company and the National Center for Advancing Translational Sciences; 2004-.). Generally, IRI compounds are dissolved in cell culture media at 4-6 concentrations, spanning past the IRI IC50 (i.e. IC₅₀=1 mM, concentrations chosen for cytotoxicity are 0.5, 1, 5 and 10 mM) to achieve cytotoxic concentrations. HEPG2 cells are cultured and transferred to 96-welled plates, where the cells are incubated with the compounds at different concentrations at 37° C. for 3 h. A no-compound row is used as our positive control, Triton-1X is used as our negative control and a no-cell control acts as our media control. The plate data is averaged and transformed to viability percentage (%) using Microsoft Excel. The LD₅₀ is determined by nonlinear regression using a 4-parameter sigmoidal dose-response fitting equation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

We claim:

1. A compound of formula (I): 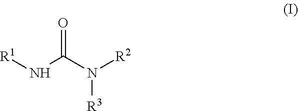
its enantiomer, diastereomer, or its physiologically acceptable salt thereof, wherein
A) R¹ is selected from the group consisting essentially of:
a) an unmodified C₁₋₈ alkyl;
b) a heteroatom-modified C₁₋₈ alkyl; and
c) 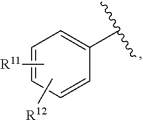
wherein R¹¹ and R¹² are selected from the group consisting essentially of:
a. H;
b. an unmodified C₁₋₄-alkyl;
c. a heteroatom-modified C₁₋₄-alkyl;
d. an unmodified C₁₋₄-alkoxy;
e. a heteroatom-modified C₁₋₄-alkoxy;
f. —N(R^{a})₂, wherein R^{a} is selected from the group consisting essentially of:
i. H;
ii. an unmodified C₁₋₄-alkyl;
iii. a heteroatom-modified C₁₋₄-alkyl;
iv. an unmodified aryl; and
v. a heteroatom-modified aryl;
g. —C(═O)R^{b}, wherein R^{b} is selected from the group consisting essentially of:
i. H;
ii. an unmodified C₁₋₄-alkyl;
iii. a heteroatom-modified C₁₋₄-alkyl;
iv. an unmodified aryl; and
v. a heteroatom-modified aryl; and
h. —CO₂R^{c}, wherein R^{c} is selected from the group consisting essentially of:
i. H;
ii. an unmodified C₁₋₄-alkyl;
iii. a heteroatom-modified C₁₋₄-alkyl;
iv. an unmodified aryl;
v. a heteroatom-modified aryl;
i. an aldehyde;
j. a halogen;
k. —CH₃;
l. isopropyl;
m. trifluoromethyl;
n. tert-butyl;
o. —C(═O)OCH₃;
p. —OCH₃;
q. —CH₂—OH;
r. —F, —Cl; and
s. —Br,
B) R² and R³ are selected from the group consisting essentially of:
a) a connected three to eight membered unmodified carbon ring that includes the nitrogen atom to which R² and R³ are bonded;
b) a connected three to eight membered heteroatom-modified carbon ring that includes the nitrogen atom to which R² and R³ are bonded; and
c) R³ being H, and R² being 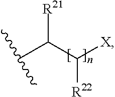
wherein
a. n is from 1 to 7,
b. R²¹ is selected from the group consistently essentially of:
i. H;
ii. —OH; and
iii. —CH₂OH,
c. R²² is selected from the group consistently essentially of:
i. H;
ii. —OH;
iii. —CH₂OH;
iv. an unmodified C₁₋₃-alkyl; and
v. a heteroatom-modified C₁₋₃-alkyl, and
d. X is selected from the group consisting essentially of:
i. —OR^{d}, wherein R²² on the carbon atom attached to X is hydrogen and R^{d} is selected from the group consisting essentially of:
1. —H;
2. a C₁₋₃-alkyl; and
3. —CH₂—CH₂OH;
ii. —N(R^{e})₂, wherein R²² on the carbon atom attached to X is hydrogen and R^{e} is selected from the group consisting essentially of:
1. —H;
2. a C₁₋₃-alkyl; and
3. —CH₂—CH₂OH;
iii. —CO₂H; and
iv. an aryl, wherein R²² on the carbon atom attached to X is hydrogen.

2. The compound of formula (I), its enantiomer, diastereomer, or its physiologically acceptable salt thereof, as recited in claim 1, wherein
R¹ is

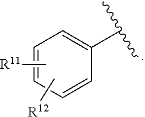

3. The compound of formula (I), its enantiomer, diastereomer, or its physiologically acceptable salt thereof, as recited in claim 2, wherein
each R¹¹ and R¹² independently is selected from the group consisting essentially of H, an unmodified C₁₋₄-alkyl, a heteroatom-modified C₁₋₄-alkyl, an unmodified C₁₋₄-alkoxy, a heteroatom-modified C₁₋₄-alkoxy, —CO₂R^{c}, and a halogen.

4. The compound of formula (I), its enantiomer, diastereomer, or its physiologically acceptable salt thereof, as recited in claim 1, wherein
each R¹¹ and R¹² independently is selected from the group consisting essentially of H, —CH₃, isopropyl, trifluoromethyl, tert-butyl, —C(═O)OCH—₃, —OCH₃, —CH₂OH, —F, —Cl, and —Br.

5. The compound of formula (I), its enantiomer, diastereomer, or its physiologically acceptable salt thereof, as recited in claim 1, wherein
the halogen is F, Cl or Br.

6. The compound of formula (I), its enantiomer, diastereomer, or its physiologically acceptable salt thereof, as recited in claim 1, wherein
R³ is H, and R² is 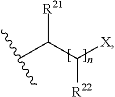
wherein
X is selected from the group consisting of —OR^{d}, —N(R^{e})₂, —CO₂H, and aryl, wherein R^{d} and each R^{e} independently is selected from the group consisting essentially of H, a C₁₋₃-alkyl, and —CH₂—CH₂OH;
R²¹ is selected from the group consisting essentially of H, —OH, and —CH₂OH;
each R²² independently is selected from the group consisting essentially of H, —OH, —CH₂OH, and C₁₋₃-alkyl, wherein when X is one from a group consisting essentially of —OR^{d}, —N(R^{e})₂, and aryl, R²² on the carbon atom attached to X is H; and
n is from 1 to 7.

7. The compound of formula (I), its enantiomer, diastereomer, or its physiologically acceptable salt thereof, as recited in claim 1, wherein
R²¹ is selected from the group consisting essentially of —H, R²² is —H and —OH, and X is —OH.

8. The compound of formula (I), its enantiomer, diastereomer, or its physiologically acceptable salt thereof, as recited in claim 1, wherein
R²¹ is selected from the group consisting essentially of —H, R²² is selected from the group consisting essentially of —H and —OH, and X is —OR^{d}, wherein R² is —CH₂—CH₂OH.

9. The compound of formula (I), its enantiomer, diastereomer, or its physiologically acceptable salt thereof, as recited in claim 1, wherein
R²¹ is —CH₂OH, R²² is selected from the group consisting essentially of —H and —OH, and X is —OH.

10. The compound of formula (I), its enantiomer, diastereomer, or its physiologically acceptable salt thereof, as recited in claim 1, wherein the compound is selected from the group consisting essentially of:

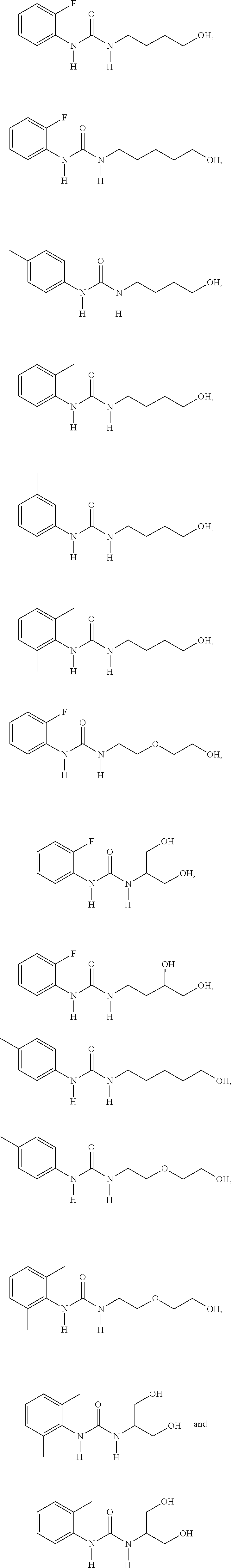

11. The compound of formula (I), its enantiomer, diastereomer, or its physiologically acceptable salt thereof, as recited in claim 1, wherein the compound is:

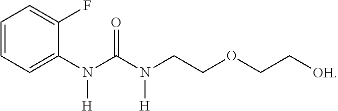 [SMILES: O=C(NCCOCCO)Nc1ccccc1F]

12. The compound of formula (I), its enantiomer, diastereomer, or its physiologically acceptable salt thereof, as recited in claim 1, wherein the compound is:

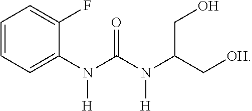 [SMILES: O=C(Nc1ccccc1F)NC(CO)CO]

13. The compound of formula (I), its enantiomer, diastereomer, or its physiologically acceptable salt thereof, as recited in claim 1, wherein the compound is:

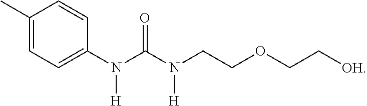 [SMILES: Cc1ccc(NC(=O)NCCOCCO)cc1]

14. The compound of formula (I), its enantiomer, diastereomer, or its physiologically acceptable salt thereof, as recited in claim 1, wherein the compound is:

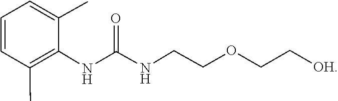 [SMILES: Cc1cccc(C)c1NC(=O)NCCOCCO]

15. The compound of formula (I), its enantiomer, diastereomer, or its physiologically acceptable salt thereof, as recited in claim 1, wherein the compound is:

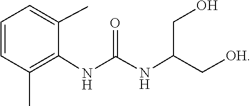 [SMILES: Cc1cccc(C)c1NC(=O)NC(CO)CO]

16. The compound of formula (I), its enantiomer, diastereomer, or its physiologically acceptable salt thereof, as recited in claim 1, wherein the compound is:

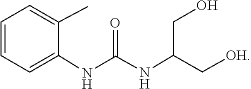 [SMILES: Cc1ccccc1NC(=O)NC(CO)CO]

17. The compound of formula (I), its enantiomer, diastereomer, or its physiologically acceptable salt thereof, as recited in claim 1, wherein the compound is:

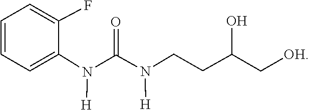 [SMILES: O=C(NCCC(O)CO)Nc1ccccc1F]

18. A composition comprising the compound of formula (I), its enantiomer, diastereomer, or its physiologically acceptable salt thereof, as recited in claim 1, and a physiologically acceptable carrier, excipient or solvent.

19. The composition of claim 18, being substantially free of dimethyl sulfoxide (DMSO) and glycerol.

20. The composition of claim 18, further comprising a cryoprotective agent or an ice crystal recrystallization inhibitor.

21. The composition of claim 18, wherein the cryoprotective agent is one from the group consisting of dimethyl sulfoxide (DMSO), trehalose, a polymer, lactobionate, glycerol, polyvinylalcohol, hydroxyethyl starch (HES), ethylene glycol (EG), propylene glycol (PG), mannitol, amino acids, a reducing sugar, a biocompatible disaccharide, and a biocompatible polysaccharide.

22. A method for cryopreserving a biological material comprising:
a) combining the biological material with a solution comprising the compound of formula (I), its enantiomer, diastereomer, or its physiologically acceptable salt thereof, as recited in claim 1;
b) cooling the combination from step a) to a storage temperature at or below the freezing point of the solution; and
c) storing the cooled combination at the storage temperature.

23. The method of claim 22, wherein the cooling comprises performing a rate-controlled cooling of about 1° C. per minute over about 16 hours.

24. The method of claim 22, wherein the biological material is cells and the combining step comprises suspending the cells in the solution and the combination is a suspension of the cells in the solution.

25. A process for preparation of the compound of formula (I), its enantiomer, diastereomer, or its physiologically acceptable salt thereof, as recited in claim 1, the process comprising the steps of:
reacting an isocyanate of formula 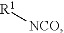
with an amine of formula 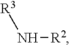
to form the compound of formula (I)

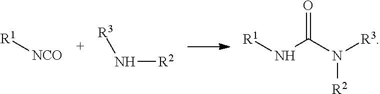

26. A process for preparation of the compound of formula (I), its enantiomer, diastereomer, or its physiologically acceptable salt thereof, as recited in claim 1, the process comprising the steps of:
coupling a first amine of formula 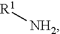
with a second amine of formula 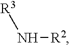
in the presence of a coupling agent to form the compound of formula (I) [chemical structure]
